(19) United States
(12) Patent Application Publication
(10) Pub. No.: US 2025/0034592 A1
(43) Pub. Date: Jan. 30, 2025

(54) NEUROPEPTIDE-EXPRESSING VECTORS AND METHODS FOR THE TREATMENT OF EPILEPSY

(71) Applicant: CHARITE - UNIVERSITATSMEDIZIN BERLIN, Berlin (DE)

(72) Inventors: Regine HEILBRONN, Berlin (DE); Christoph SCHWARZER, Innsbruck (AT)

(73) Assignee: CHARITE - UNIVERSITATSMEDIZIN BERLIN, Berlin (DE)

(21) Appl. No.: 18/760,115

(22) Filed: Jul. 1, 2024

Related U.S. Application Data
(62) Division of application No. 16/310,718, filed on Dec. 17, 2018, now abandoned.

(30) Foreign Application Priority Data
Jun. 16, 2016 (EP) 16174827.2

(51) Int. Cl. C12N 15/86; A61P 25/08; C07K 14/665

(52) U.S. Cl. CPC C12N 15/86; A61P 25/08; C07K 14/665; C12N 2750/14141

(57) ABSTRACT

The present invention provides delivery vectors for transferring a nucleic acid sequence to a cell in vitro, ex vivo or in vivo. The present invention provides methods of delivering a nucleic acid sequence to a cell and methods of treating focal epilepsies.

### SEQUENCE LISTING

The instant application contains a Sequence Listing which has been filed electronically in ASCII format and is hereby incorporated by reference in its entirety. Said ASCII copy, created on Sep. 19, 2024, is named C75042WOUS-A SEQ LIST.xml and is 37,205 bytes in size.

The present invention provides delivery vectors for transferring a nucleic acid sequence that encodes a pre-propeptide to a cell in vitro, ex vivo or in vivo. The present invention provides methods of delivering a nucleic acid sequence to a cell and methods of treating focal epilepsies.

With a prevalence of 1-2%, epilepsies belong to the most frequent neurological diseases worldwide (McNamara et al., 1999). Mesial temporal lobe epilepsy (mTLE) is the most frequent type of epilepsy in humans and is frequently induced by traumatic brain injury. Hippocampal sclerosis and accompanying neurological deficits are key features of mTLE (for review, see Engel et al., 2001). Despite the introduction of a plethora of antiepileptic drugs over the last few decades, the rate of drug-resistant epilepsies (30% to 70%) did not improve since the study of Coatsworth in 1971 (Coatsworth et al., 1971, Loscher et al., 2011). To date, surgical resection of the epileptogenic focus remains as the ultimate option for some of these patients. Even then, in certain subgroups of patients, less than 50% remain seizure free for at least one year after removal of the epileptic focus (Spencer et al., 2008).

Since the early 1980s, there has been evidence that opioids, namely dynorphin (Dyn), act as modulators of neuronal excitability in vitro (Henriksen et al., 1982, Siggins et al., 1986). In line with this, the deletion of the prodynorphin (pDyn) coding sequence in mice (Loacker et al., 2007) and low Dyn levels in humans due to mutations in the promoter region (Stogmann et al., 2002, Gambardella et al., 2003) are associated with increased vulnerability to the development of epilepsy. In most animal models of temporal lobe epilepsy (TLE; comprising epilepsies arising cortical=lateral TLE and mTLE), cortical and hippocampal pDyn expression is reduced after an initial, short peak of over-expression (for review, see (Simonato et al., 1996, Schwarzer et al., 2009). This is in line with most probably short-lived post-ictally increased pDyn mRNA in hippocampal granule cells (Pirker et al., 2009) accompanied by an overall reduction of Dyn-immunoreactivity in surgically removed tissue obtained from mTLE patients (de Lanerolle et al., 1997).

Dynorphins act preferentially on kappa opiod receptors (KOR). Despite the reduction of endogenous Dyn, KOR remain available as drug target under epileptic conditions, and the application of KOR agonists can suppress experimental seizures (Tortella et al., 1988, Takahashi et al., 1990, Solbrig et al., 2006, Loacker et al., 2007, Zangrandi et al. 2016). Various selective KOR applied through different routes yielded time- and dose-dependent effects similar to those upon treatment with phenytoin or phenobarbital in models of epilepsy (for review, see (Simonato et al., 1996). We recently demonstrated that activation of KOR promotes the survival of hippocampal and amygdala neurons subsequent to the acute phase after unilateral injection of kainic acid in mice (Schunk et al., 2011).

The object of the present invention is to provide delivery vectors for transferring a nucleic acid sequence encoding a pre-propeptide or a peptide comprising a signal sequence that enables the packing of said pro-peptide, e.g. a prodynorphin into vesicles, wherein the pro-peptide undergoes maturation and the active substance which is a dynorphin or variant of dynorphin is released upon a frequency of action potentials that exceeds a certain excitation threshold. The object of the present invention is, thus, to provide delivery vectors for transferring a nucleic acid sequence to a cell in vitro, ex vivo or in vivo. Object of the invention is in particular a vector-based therapy for treatment of focal epilepsies with pre-prodynorphin or dynorphin or variants thereof. The inventive delivery vectors comprising a nucleic acid encoding pre-prodynorphin or prodynorphin or dynorphin or variants thereof shall transduce neurons, release pre-prodynorphin or prodynorphin or dynorphin or variants thereof and thus provide activation of KOR in the epileptogenic focus, thereby inhibiting seizures.

Subject of the present invention is a delivery vector comprising a DNA sequence encoding pre-prodynorphyin or pre-prodynorphin-variants and wherein said delivery vector drives expression of pre-prodynorphyin or pre-prodynorphin-variants as pre-propeptides comprising a signalpeptide whereas said pre-prodynorphyin or pre-prodynorphin-variants comprise at least one of the following sequences selected from the group:
a. Dyn A that is SEQ Id No. 7 (AA 207-223 of SEQ ID No. 1; ppDyn) or a variant thereof consisting of the first 13 AA (first from the N-terminal end) or a variant thereof consisting of the first 8 AA (first from the N-terminal end)
b. Dyn B that is SEQ ID No. 8 (AA 226-238 of SEQ ID No. 1; ppDyn)
c. leumorphin that is SEQ ID No. 9 (AA 226-254 of SEQ ID No. 1; ppDyn)
d. variants of Dyn A according to SEQ Id No.7 having an amino acid sequence identity of at least 60% within the first 8 AA counted from the N-terminus of SEQ ID No. 7 (YGGFLRRI (SEQ ID NO: 18)) i.e. having an amino acid sequence identity of at least 60% within the sequence YGGFLRRI (SEQ ID NO: 18) comprised in SEQ ID No. 7.
e. variants of Dyn B according to SEQ ID No. 8 having an amino acid sequence identity of at least 60% within the first 8 AA counted from the N-terminus of SEQ ID No. 8 (YGGFLRRQ (SEQ ID NO: 19)) i.e. having an amino acid sequence identity of at least 60% within the sequence YGGFLRRQ (SEQ ID NO: 19) comprised in SEQ ID No. 8.
f. variants of leumorphin according to SEQ ID No. 9 having an amino acid sequence identity of at least 60% within the first 8 AA counted from the N-terminus of SEQ ID No. 9 (YGGFLRRQ (SEQ ID NO: 19)), i.e. having an amino acid sequence identity of at least 60% within the sequence YGGFLRRQ (SEQ ID NO: 19) comprised in SEQ ID No. 9.

60% sequence identity is defined as follows: 3 of the first 8 N-terminal amino acids may be removed or replaced by another amino acid. Percentage of sequence identity is calculated for the shortened peptide in case of truncated peptide variants. Introduction of additional amino acids are handled as gap in the original sequence, deletions are handled as gap in the modified peptide for calculation of sequence identity (YGGFLRRQ (SEQ ID NO: 19) differs from YG-FLRRQ (SEQ ID NO: 20) only by 1 AA, although now AA in positions 3, 4, 5, 6 and 7 are different). In any case a variant of SEQ ID No. 7 having an amino acid sequence identity of at least 60% from the N-terminus in the first 8 AA may be a variant that comprises the sequence: YGZFLRKZ (SEQ ID NO: 21) with Z standing for any amino acid and K resubstituting R in position 7, conserving the peptidase recognition site.

Throughout the application “Z” in an amino acid sequence stands for any of the naturally occurring amino acids, in a specific embodiment “Z” may be selected from the group comprising alanine, glycine, asparagine, glutamine, leucine, serine, valine and isoleucine.

According to the present invention said delivery vector comprises a DNA sequence encoding the pre-propeptide of dynorphin or dynorphin-variants. This means said vector comprises a DNA sequence encoding a signal peptide. As one aspect, the present invention provides delivery vectors for transferring a nucleic acid to a cell, the delivery vector comprising a segment encoding a secretory signal peptide. The DNA sequence encoding the signal peptide may be a sequence according to SEQ ID No.: 4. In another aspect of the invention said delivery vector comprises a DNA sequence encoding a propeptide fragment. In a particular aspect of the invention said propeptide fragment is a sequence according to SEQ ID No.5.

The advantage of the present delivery vectors is a release on demand of dynorphins or dynorphin-variants. This means that prodynorphin (or a variant of prodynorphin) is packed into vesicles, undergoes maturation and is released on demand upon high frequency stimulation (e.g. stimulation ≥8 Hz) as it occurs at seizure onset (see FIG. 4). A release-on-demand formulation, thus, provides a pre-prodynorphin (or a variant of pre-prodynorphin) that is then packed into vesicles, undergoes maturation and the active substance which is a dynorphin or variant of dynorphin is released upon a frequency of action potentials that exceeds a certain threshold. Said certain threshold maybe a threshold that is ≥6 Hz, in another embodiment ≥7 Hz in another embodiment ≥8 Hz, in another embodiment ≥9 Hz. This means said release-on-demand is triggered by increased neuronal firing frequency. Said increased neuronal firing frequency maybe measured by EEG (electroencephalography), increased means a value measured by EEG in said subject that is ≥6 Hz, in another embodiment ≥7 Hz in another embodiment ≥8 Hz, in another embodiment ≥9 Hz.

This means that the present delivery vectors drive expression of pre-propeptides that enable the provision of dynorphin or dynorphin-variants on demand as such delivery vectors at first express the pre-propeptides in neurons, where the resulting propeptides are sorted into large dense core vesicles, where it is enzymatically processed and the derived peptides are stored until a sufficiently intense excitation leads to their release, i.e. said release is triggered by increased neuronal firing frequency as explained above. Dyn peptides bind to pre- and/or postsynaptic KOR which activate G-proteins, which, beside others, regulate ion channels to dampen further amplification and spread of neuronal excitation. The translation of the signal peptide of ppDyn is the initial step of the sorting of prodynorphin expressed in neurons into “large dense core” vesicles (LDV). Using existing mechanisms in neurons, the prodynorphin is enzymatically processed to mature peptides and transported to axon terminals. LDV are stored in the axon terminals and released in a stimulation-dependent manner. High frequency stimulation as explained above, like at the onset of seizures, induce the release, while low-frequency stimulation does not. This creates a drug on demand situation. Released Dyn peptides bind to pre- and/or postsynaptic KOR, which activate G-proteins, that, beside others, regulate ion channels to dampen further amplification and spread of neuronal excitation.

In other words a release-on-demand composition is a composition that releases the peptide having agonistic effects on human KOR derived from any of the delivery vectors or recombinant virus particles or liposomes according to the present invention at the onset of seizure in said subject. The onset of seizure may be characterized by increased neuronal firing frequency that maybe measured by EEG (electroencephalography), wherein increased means a value measured by EEG in said subject that is ≥6 Hz, in another embodiment ≥7 Hz in another embodiment ≥8 Hz, in another embodiment ≥9 Hz.

A variant of SEQ ID No. 7 having an amino acid sequence identity of at least 60% from the N-terminus in the first 8 AA is a variant that has an amino acid sequence identity of at least 60% in the sequence of: YGGFLRRI (SEQ ID NO: 18). Sequence identity is defined as follows: 3 of the first 8 amino acids may be removed or replaced by another amino acid. In any case a variant of SEQ ID No. 7 having an amino acid sequence identity of at least 60% from the N-terminus in the first 8 AA may be a variant that comprises the sequence: YG-FLRKZ (SEQ ID NO: 22), where “-” stands for a deleted AA, while Z stands for any AA.

A variant of SEQ ID No. 8 having an amino acid sequence identity of at least 60% from the N-terminus in the first 8 AA is a variant that has an amino acid sequence identity of at least 60% in the sequence of YGGFLRRQ (SEQ ID NO: 19) Sequence identity is defined as follows: 3 of the first 8 amino acids may be removed or replaced by another amino acid. In any case a variant of SEQ ID No. 8 having an amino acid sequence identity of at least 60% from the N-terminus in the first 8 AA may be a variant that comprises the sequence: YGGFLZZZ (SEQ ID NO: 23), with Z standing for any amino acid.

A variant of SEQ ID No. 9 having an amino acid sequence identity of at least 60% from the N-terminus in the first 8 AA is a variant that has an amino acid sequence identity of at least 60% in the sequence of YGGFLRRQ (SEQ ID NO: 19). Sequence identity is defined as follows: 3 of the first 8 amino acids may be removed or replaced by another amino acid. In any case a variant of SEQ ID No. 9 having an amino acid sequence identity of at least 60% from the N-terminus in the first 8 AA may be a variant that comprises the sequence: YGAFLRZA (SEQ ID NO: 24), with Z standing for any amino acid.

In a specific embodiment subject of the invention is a delivery vector as above described, wherein the variants have an amino acid sequence identity of at least 70% from the N-terminus in the first 8 AA of SEQ ID No. 7, SEQ ID No. 8 or SEQ ID No. 9, respectively. 70% sequence identity means that up to 2 of the first 8 amino acids may be removed or replaced by another amino acid.

In a specific embodiment subject of the invention is a delivery vector as above described, wherein the variants have an amino acid sequence identity of at least 80% from the N-terminus in the first 8 AA of SEQ ID No. 7, SEQ ID No. 8 or SEQ ID No. 9, respectively. 80% sequence identity means that 1 of the first 8 amino acids may be removed or replaced by another amino acid.

In a specific embodiment subject of the invention is a delivery vector as above described, wherein said delivery vector comprises a DNA sequence encoding a pre-prodynorphin-variant that comprises at least one of the following sequences of variants selected from the group: SEQ ID No. 10, SEQ ID No. 11 and SEQ ID No. 12 wherein Z may be any amino acid and wherein preferably in SEQ ID No. 10 at least one Z is different in comparison to the sequence SEQ ID No. 7, and wherein preferably in SEQ ID No. 11 at least one Z is different in comparison to the sequence SEQ ID No. 8, and wherein preferably in SEQ ID No. 12 at least one Z is different in comparison to the sequence SEQ ID No. 9.

In a specific embodiment subject of the invention is a delivery vector as above described, wherein said delivery vector comprises multiple DNA sequences encoding SEQ ID No. 7, SEQ ID No. 8 and/or SEQ ID No. 9 or variants thereof wherein the sequences according to SEQ ID No. 7, SEQ ID No. 8 and/or SEQ ID No. 9 or variants thereof are flanked by peptidase recognition signals.

This means as an example that said delivery vector may comprise a DNA sequence encoding SEQ ID No. 7 two times in a way that two molecules of a peptide according to SEQ ID No.7 would be derived from one delivery vector.

Peptidase (prohormone convertase) recognition signals are known to a person skilled in the art and may be single or paired basic amino acids, preferably but not exclusively K, R, KR, RK or RR.

In a specific embodiment subject of the invention is a delivery vector as above described, wherein said delivery vector comprises multiple DNA sequences encoding SEQ ID No. 8 and/or SEQ ID No. 9 or variants thereof wherein the sequences according, SEQ ID No. 8 and/or SEQ ID No. 9 or variants thereof are flanked by peptidase recognition signals.

In a specific embodiment subject of the invention is a delivery vector as above described, wherein said delivery vector comprises multiple DNA sequences encoding SEQ ID No. 8 and/or SEQ ID No. 9 or variants thereof and wherein said delivery vector does not comprise DNA encoding SEQ ID No. 6 and/or 7.

In a specific embodiment subject of the invention is a delivery vector as above described, wherein said delivery vector comprises a DNA encoding SEQ ID No. 2 or SEQ ID No. 14.

In a specific embodiment subject of the invention is a delivery vector as above described, wherein said delivery vector comprises a DNA encoding SEQ ID No. 3 or SEQ ID No. 15.

The delivery vectors produced according to the present invention are useful for the delivery of nucleic acids to cells in vitro, ex vivo, and in vivo. In particular, the delivery vectors can be advantageously employed to deliver or transfer nucleic acids to animal, more preferably mammalian, cells.

Suitable vectors include viral vectors (e.g., retrovirus, lentivirus, alphavirus; vaccinia virus; adenovirus, adeno-associated virus, or herpes simplex virus), lipid vectors, polylysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Birnaviridac; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Lutcovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picomaviridae; Plasmaviridae; Prodoviridae; Polydnaviridae; Potexvirus group; Potyvirus; Poxviridac; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Tobovirus; Totiviridae; Group Tymovirus; and Plant virus satellites. Protocols for producing recombinant viral vectors and for using viral vectors for nucleic acid delivery can be found in (Ausubel et al., 1989) and other standard laboratory manuals (e.g., Rosenzweig et al. 2007). Particular examples of viral vectors are those previously employed for the delivery of nucleic acids including, for example, retrovirus, lentivirus, adenovirus, adeno-associated virus (AAV) and other parvoviruses, herpes virus, and poxvirus vectors. The term “parvovirus” as used herein encompasses the family Parvoviridae, including autonomous parvoviruses, densoviruses and dependoviruses. The term AAV includes all vertebrate variants especially of human, primate, other mammalian, avian or serpentine origin. The autonomous parvoviruses include members of the genera Parvovirus, Erythrovirus, Bocavirus, Densovirus, Iteravirus, and Contravirus. Exemplary autonomous parvoviruses include, but are not limited to, minute virus of mice, bovine parvovirus, canine parvovirus, chicken parvovirus, feline panleukopenia virus, feline parvovirus, goose parvovirus, HI parvovirus, muscovy duck parvovirus, bocavirus, bufavirus, tusavirus and B19 virus, and any other virus classified by the International Committee on Taxonomy of Viruses (ICTV) as a parvovirus. Other autonomous parvoviruses are known to those skilled in the art. See, e.g. (Berns et al. 2013).

In one embodiment of the invention said delivery vector comprises in addition a recombinant adeno-associated virus (AAV) vector genome or a recombinant lentivirus genome.

In one particular embodiment of the invention said delivery vector comprises in addition a recombinant AAV vector, wherein preferably said vector is a serotype of human or primate origin.

In one particular embodiment of the invention said delivery vector comprises in addition a recombinant adeno-associated virus (AAV) vector genome, wherein said vector is a human serotype vector selected from the group comprising serotypes 1, 2, 3, 4, 5, 6, 7, 8, 9, 10, rh10, 11, 12, 13, 14, serpentine AAV, ancestral AAV, or AAV capsid mutants derived thereof, preferably but not exclusively of AAV serotype 1 or 2.

In one particular embodiment of the invention said delivery vector is a single stranded AAV vector or a self-complimentary (or dimeric) duplex vector.

In one particular embodiment of the invention said delivery vector is a delivery vector as described above, wherein the DNA sequence encoding pre-prodynorphyin or pre-prodynorphin-variants is operatively linked to expression control elements comprising a promoter and/or enhancer that produces sufficient expression of the gene product of interest to obtain a therapeutic effect.

For example, the encoding nucleic acid may be operably associated with expression control elements, such as transcription/translation control signals, origins of replication, polyadenylation signals, and internal ribosome entry sites (IRES), promoters, enhancers, and the like. It will further be appreciated that a variety of promoter/enhancer elements may be used depending on the level and tissue-specific expression desired. The promoter/enhancer may be constitutive or inducible, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced. Promoter/enhancer elements that are functional in the target cell or subject to be treated are most preferred. Mammalian promoter/enhancer elements are also preferred. Most preferred are promoter/enhancer elements active in human neurons and not, or to a lesser extend in glial cells. The promoter/enhancer element may express the transgene constitutively or inducibly.

Exemplary constitutive promoters include, but are not limited to a Beta-actin promoter, a cytomegalovirus promoter, a cytomegalovirus-enhancer/chicken beta-actin hybrid promoter, and a Rous sarcoma virus promoter. Inducible expression control elements are generally employed in those applications in which it is desirable to provide regulation over expression of the heterologous nucleic acid sequence(s). Inducible promoters/enhancer elements for gene delivery include neuron-specific, brain-specific, muscle specific (including cardiac, skeletal and/or smooth muscle), liver specific, bone marrow specific, pancreatic specific, spleen specific, and lung specific promoter/enhancer elements. In particular embodiments, the promoter/enhancer is functional in cells or tissue of the CNS, and may even be specific to cells or tissues of the CNS. Such promoters/enhancers include but are not limited to promoters/enhancers that function in the eye (e.g., retina and cornea), neurons (e.g., the neuron specific enolase, AADC, synapsin, or serotonin receptor promoter), glial cells (e.g., S100 or glutamine synthase promoter), and oligodendrocytes. Other promoters that have been demonstrated to induce transcription in the CNS include, but are not limited to, myelin basic protein (MBP) promoter (Tani et al., 1996), and the prion promoter (Loftus et al., 2002). Preferred is a neuron-specific promoter displaying significantly reduced, preferably no expression in glial cells.

Other inducible promoter/enhancer elements include drug-inducible, hormone-inducible and metal-inducible elements, and other promoters regulated by exogenously supplied compounds, including without limitation, the zinc-inducible metalothionein (MT) promoter; the dexamethasone (Dex)-inducible mouse mammary tumor virus (MMTV) promoter; the T7 polymerase promoter system (see WO 98/10088); the ecdysone-inducible insect promoter (No et al, 1996); the tetracycline-repressible system (Gossen and Bujard, 1992); the tetracycline-inducible system (Gossen et al., 1995); see also (Harvey et al., 1998); the RU486-inducible system (Wang, DeMayo et al., 1997); (Wang, Xu et al., 1997); and the rapamycin-inducible system (Magari et al., 1997).

In a particular embodiment of the invention the promoter and/or enhancer is selected from the group comprising constitutively active promoters e.g. CMV (cytomegalovirus immediate-early gene enhancer/promoter)- or CBA promoter (chicken beta actin promoter and human cytomegalovirus IE gene enhancer), or inducible promoters comprising Gene Switch, tet-operon derived promotor, or neuron-specific promoters derived of e.g. phosphoglycerate kinase (PGK), synapsin-1 (SYN), neuron-specific enolase (NSE), preferably but not exclusively of human origin.

In a particular embodiment of the invention said delivery vector further comprises a posttranscriptional regulatory element, preferably the woodchuck-hepatitis-virus-posttranscriptional-regulatory element. Other possible posttranscriptional regulatory elements are known to a person skilled in the art.

Subject of the present invention is furthermore a recombinant gene therapy vector comprising the foreign, therapeutic coding sequence, which is flanked by genetic elements for its expression and by virus-specific cis elements for its replication, genome packaging, genomic integration etc. The said virus genome is encapsidated as virus particle consisting of virus-specific proteins as in the case of AAV. In the case of lentivirus vectors the viral genome and virus-specific proteins, like reverse transcriptase and others are encapsidated into lentivirus capsids. These are enveloped by a lipid bilayer into which virus-specific proteins are embedded. Liposomes comprise the above described nucleotide sequences or entire DNA backbones including all regulatory elements of the gene therapy-, or delivery vector.

Examples of liposomes include DSPC (1,2-distearoyl-sn-glycero-3-phosphocholine, cholesterol, DSPE-PEG2000 (1,2-distearoyl-sn-glycero-3-phosphoethanol-amine-N-[amino (polyethylene glycol)-2000], or DSPE-PEG2000-mal (1,2-distearoyl-sn-glycero-3-phosphoethanol-amine-N-[maleimide (polyethylene glycol)-2000] or variants comprising sphingomyelin/cholesterol and phosphatidic acid.

In one particular embodiment of the invention said delivery vector comprises in addition a recombinant adeno-associated virus (AAV) vector genome and said recombinant AAV (rAAV) vector genome is encapsidated in an AAV capsid.

Adeno-associated viruses (AAV) have been developed as nucleic acid delivery vectors. For a review, see (Muzyczka, 1992). AAV are helper-dependent parvoviruses requiring a helper virus, typically adenovirus or herpesvirus for productive replication. AAV represent a growing family of currently 14 naturally occurring serotypes of human or primate origin. AAVs of other mammalian species, or of avian or insect origin have been described (see Berns et al., 2013). The AAVs have small icosahedral capsids, 18-26 nanometers in diameter and contain a single-stranded DNA genome of 4-5 kilobases in length. AAV encapsidates both AAV DNA strands, either the sense or antisense DNA strand is incorporated into one virion. The AAV genome carries two major open reading frames encoding the genes rep and cap. Rep encodes a family of overlapping, nonstructural, regulatory proteins. In the best-studied AAV prototype strain, AAV2, the mRNAs for Rep78 and Rep68 are transcribed from the AAV p5 promoter (Stutika et al. 2015). Rep78/68 are required for AAV transcription, AAV DNA replication, AAV integration into the host cell genome and its rescue therefrom. Rep52 and Rep40 represent N-terminally truncated versions of Rep78 and Rep68 transcribed from a separate promoter, p19 and are required for encapsidation of the newly synthesized AAV genome into preformed AAV capsids. These are formed by the three cap gene-derived proteins, VP1, VP2, and VP3. The cap ORF also encodes AAP, an assembly-enhancing protein that does not form part of the capsid. The AAV ORFs are flanked by inverted terminal repeat sequences (ITRs) at either end of the genome. These vary in length between AAV serotypes, in AAV2 these comprise around 145 bp, the first 125 bp thereof are capable of forming Y- or T-shaped duplex structures. The ITRs represent the minimal AAV sequences required in cis for DNA replication, packaging, genomic integration and rescue. Only these have to be retained in an AAV vector to ensure DNA replication and packaging of the AAV vector genome. Foreign genes flanked by AAV-ITRs will be replicated and packaged into AAV capsids provided the AAV genes rep and cap are expressed in trans in the chosen packaging cell (Muzyczka, 1992).

AAV are among the few viruses that can persist over months and years in non-dividing cells in vivo, including neurons, muscle, liver, heart and others. Wildtype AAV2 has been shown to integrate its genome into the host cell genome in a Rep78/68-dependent manner, with a preference for chromosomal loci with DNA sequence homology to the so-called Rep-binding site which forms part of the AAV-ITRs (Huser et al., 2014). In contrast, AAV vectors mostly persist as nuclear episomes. Devoid of the AAV genes rep and cap AAV vectors rarely integrate at all, and if so without genomic preference (Hüser et al., 2014). Nonetheless long-term AAV persistence has been shown in non-dividing, post mitotic cells including neurons which renders AAV vectors ideal for CNS transduction and long-term gene addition therapy of chronic diseases of genetic or acquired origin.

Generally, a recombinant AAV vector (rAAV) genome will only retain the inverted terminal repeat (ITR) sequence(s) so as to maximize the size of the transgene that can be efficiently packaged by the vector. The structural- and non-structural protein-coding sequences may be provided in trans, e.g., from a vector, such as a plasmid, by stably integrating the respective genes into a packaging cell, or in a recombinant helper virus such as HSV or baculovirus, as reviewed in (Mietzsch, Grasse et al., 2014). Typically, the rAAV vector genome comprises at least one AAV terminal repeat, more typically two AAV terminal repeats, which generally will be at the 5′ and 3′ ends of the heterologous nucleotide sequence(s). The AAV ITR may be from any AAV including serotypes 1-14. Since AAV2-derived ITRs can be crosspackaged into virtually any AAV serotype capsids, AAV2 ITRs combined with AAV2 rep are mostly employed. The AAV terminal repeats need not maintain the wild-type terminal repeat sequence (e.g., a wild-type sequence may be altered by insertion, deletion, truncation or missense mutations), as long as the terminal repeat mediates the desired functions, e.g., replication, virus packaging, integration, and/or provirus rescue, and the like. The rAAV vector genome is generally between 80% to about 105% of the size of the wild-type genome and comprises an appropriate packaging signal as part of the AAV-ITR. To facilitate packaging into an AAV capsid, the entire vector genome is preferably below 5.2 kb, more preferably up to 4.8 kb in size to facilitate packaging of the recombinant genome into the AAV capsid. So-called dimeric or self-complementary AAV vectors (dsAAV) were developed that package double-stranded instead of single-stranded AAV genomes (McCarty et al., 2001). These lead to enhanced AAV gene expression, however at the price of reduced transgene capacity. Only up to 2 kb of foreign genes can be packaged, which is enough for small genes or cDNAs including those for neuropeptides.

Any suitable method known in the art can be used to produce AAV vectors expressing the nucleic acids of this invention. AAV vector stocks can be produced by co-transfection of plasmids for the ITR-flanked AAV vector genome expressing the transgene together with an AAV rep/cap expressing plasmid of the desired serotype and adenovirus-derived helper genes for AAV replication (Grimm et al., 2003; Xiao et al., 1998). AAV vectors can also be produced in packaging cell lines of mammalian or insect origin and/or in combination with recombinant helperviruses, such as adenovirus, herpes simplex virus (HSV), another member of the herpesvirus family, or baculovirus, as reviewed and discussed in (Mietzsch, Grasse et al., 2014).

Another embodiment of the present invention is a method of delivering a nucleic acid to a cell of the central nervous system, comprising contacting the cell with the delivery vector or recombinant virus particle as described above under conditions sufficient for the DNA sequence encoding pre-prodynorphyin or pre-prodynorphin-variants to be introduced into the cell.

The delivery vectors of the present invention provide a means for delivering nucleic acid sequences into cells of the central nervous system, preferably neurons. The delivery vectors may be employed to transfer a nucleotide sequence of interest to a cell in vitro, e.g., to produce a polypeptide in vitro or for ex vivo gene therapy. The vectors are additionally useful in a method of delivering a nucleotide sequence to a subject in need thereof. In this manner, the polypeptide may thus be produced in vivo in the subject. The subject may be in need of the polypeptide because the subject has a deficiency of the polypeptide, or because the production of the polypeptide in the subject may impart some therapeutic effect, as a method of treatment or otherwise, and as explained further below.

In one particular embodiment of the method of delivering a nucleic acid to a cell of the central nervous system the pre-prodynorphyin or pre-prodynorphin-variant is produced and released from the cell.

In one particular embodiment of the method of delivering a nucleic acid to a cell of the central nervous system the method comprises contacting the cell with the recombinant virus particle or liposome as described above under conditions sufficient for the DNA sequence encoding pre-prodynorphyin or pre-prodynorphin-variants to be introduced into the cell. Conditions sufficient for the DNA sequence encoding pre-prodynorphyin or pre-prodynorphin-variants to be introduced into the cell is the contacting of the AAV capsid to host cell surface receptors and coreceptors. AAV1 capsids bind to 2-3 sialic acid linked to N-acetylgalactosamine, followed by 1-4-linked N-acetylglucosamine, whereas AAV2 capsids bind to heparin sulfate proteoglycan particularly 6-O- and N-sulfated heparins on the cell surface (Mietzsch, Broecker et al., 2014). AAV coreceptors include FGFR-1, Integrin aVb5, hepatocyte growth factor receptor (c-met) and a recently identified, universal AAV receptor, AAVR necessary for transduction with AAV1, AAV2 and others irrespective of the presence of specific glycans (Pillay et al., 2016). AAVR directly binds to AAV particles and helps trafficking to the trans Golgi network. How AAV enters the nucleus is only insufficiently understood. In any case AAV vectors are expressed in the cell nucleus.

One embodiment of the invention is a delivery vector or recombinant virus particle or liposome as described above for use as medicament.

One embodiment of the invention is a delivery vector or recombinant virus particle or liposome as described above for use the preparation of a medicament.

One embodiment of the invention is a method of treating a diseased subject in need of therapy by administering a delivery vector or recombinant virus particle or liposome as described above.

One embodiment of the invention is a delivery vector or recombinant virus particle or liposome as described above for use of treating focal epilepsy in a subject, in particular mesial temporal lobe epilepsy. One embodiment of the invention is a delivery vector or recombinant virus particle or liposome as described above for use in the preparation of a medicament for treating focal epilepsy in a subject, in particular mesial temporal lobe epilepsy. One embodiment of the invention is a delivery vector or recombinant virus particle or liposome as described above for use in preventing epileptic seizures in a subject that suffers from focal epilepsy whereby said delivery vector or recombinant virus particle or liposome provides activation of human Kappa Opioid Receptors in the epileptogenic focus, thereby inhibiting seizures. In particular said delivery vector or recombinant virus particle or liposome leads to on-demand release of peptides with agonistic effects on human Kappa Opiod Receptors in the epileptogenic focus. Said peptide(s) with agonistic effects on human Kappa Opiod Receptors are in one embodiment selected from the group of peptides having the sequence SEQ ID No.s 10, SEQ ID No.s 11, SEQ ID No.s 12, SEQ ID No.s 13, SEQ ID No.s 14 or SEQ ID No.s 15.

One embodiment of the invention is a method of treating a diseased subject in need of therapy by administering a delivery vector or recombinant virus particle or liposome as described above wherein said disease is focal, in particular mesial temporal lobe epilepsy.

One embodiment of the invention is a delivery vector or recombinant virus particle or liposome as described above for use in treating focal epilepsy or preventing epileptic seizures in a subject that suffers from focal epilepsy, in particular mesial temporal lobe epilepsy, in a subject, wherein said vector is suitable for transduction of neuronal cells of the central nervous system, e.g. brain.

One embodiment of the invention is a delivery vector or recombinant virus particle or liposome as described above for preparation of a medicament for treating focal epilepsy, in particular mesial temporal lobe epilepsy, in a subject, wherein said vector is suitable for transduction of neuronal cells of the brain.

A vector is suitable for transduction of neuronal cells of the brain means that it attaches to neuronal cell surface receptors and penetrates the cell membrane e.g. by endocytosis.

In a particular embodiment said vector or recombinant virus particle is suitable for peripheral administration or for intracranial or for intracerebral or for intrathecal administration. This is achieved by the aid of a catheter, injection needle or the like, guided by deep brain electrodes to detect the epileptogenic focus.

One embodiment of the invention is a pharmaceutical composition comprising a delivery vector or recombinant virus particle as described above and optionally a pharmaceutically acceptable carrier. Pharmaceutically acceptable carriers are known to a person skilled in the art. Pharmaceutically acceptable carrier may be nanoparticles, liposomes, nanogels, implants releasing vector particles or vector-producing cells.

Another embodiment of the present invention is a cell infected [in vitro or ex vivo] with a delivery vector or recombinant virus or liposome particle as described above. For instance the delivery vector or recombinant virus or liposome particle as described above may infect (stem) cells ex vivo during preparation of primary cell culture, or (stem) cells kept in culture. Both, primary cells or stem cells may be derived of the patient to be treated or stem from another subject, or an animal species. Infected cells are transplanted into the focus of the diseased tissue in order to produce the therapeutic peptides.

Subject of the present invention is a method of treating a subject with focal epilepsy comprising administering a delivery vector, a recombinant virus particle, or liposome or a pharmaceutical composition as described above to the subject in need thereof.

Subject matters of the present invention are furthermore peptides with agonistic effects on human Kappa Opiod Receptors (KOR) derived or derivable from any of the delivery vectors as described above.

Preferably, said peptides with agonistic effects on KOR are displaying a pKi of 7 or higher for human KOR, measured as described by (Toll et al., 1998).

Preferably, said KOR agonistic peptides displaying pKi of 7 or higher for human KOR exhibit lower pKis for human MU Opioid Receptor (MOR) and human Delta Opioid Receptor (DOR), measured as described by (Toll et al., 1998) Lower pKis for human MOR and human DOR means pKi of 6 or less.

Preferably, said KOR agonistic peptides are displaying pKi of 7.5 or higher for human KOR with lower pKis for human MOR and human DOR, measured as described by (Toll et al., 1998) Lower pKis for human MOR and human DOR means pKi of 6 or less.

In a particular embodiment peptides with agonistic effects on human Kappa Opiod Receptors (KOR) are selected from the group comprising peptides of SEQ ID No. 6, SEQ ID No. 7, SEQ ID No. 8, SEQ ID No. 9, SEQ ID No. 10, SEQ ID No. 11, and SEQ ID No. 12.

### Description of the Below Sequences:

SEQ ID No. 1 (ppDyn)
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLKELEKSK FLPSISTKEN
TLSKSLEEKL RGLSDGFREG AESELMRDAQ LNDGAMETGT
LYLAEEDPKE QVKRYGGFLR KYPKRSSEVA GEGDGDSMGH
EDLYKRYGGF LRRIRPKLKW DNQKRYGG FLRRQFKVVT
RSQEDPNAYS GELFDA

Human pre-prodynorphin before processing as expressed in the human brain.

SEQ ID No. 2 Variant 1 of ppDyn
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLKELEKSK FLPSISTKEN
TLSKSLEEKL RGLSDGFREG AESELMRDAQ LNDGAMETGT
LYLAEEDPKE QVKRYGGFLR RQFKVVTRSQ EDPNAYSGEL
FDAKRSSEVA GEGDGDSMGH EDLYKRYGGF LRRIRPKLKW
DNQKRYGG FLRRQFKVVT RSQEDPNAYS GELFDA

Human pre-prodynorphin as in SEQ ID No. 1 engineered so that the peptide neo-endorphin is removed and replaced by a second copy of leumorphin.

SEQ ID No. 3 Variant 2 of ppDyn
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLKELEKSK FLPSISTKEN
TLSKSLEEKL RGLSDGFREG AESELMRDAQ LNDGAMETGT
LYLAEEDPKE QVKRYGGFLR RQFKVVTRSQ EDPNAYSGEL
FDAKRSSEVA GEGDGDSMGH EDLYKRYGGF LRRQFKVVTR
SQEDPNAYSG ELFDAKRYGG FLRRQFKVVT RSQEDPNAYS
GELFDA

Human pre-prodynorphin variant as in SEQ ID No. 2 engineered so that the peptide Dyn A is removed and replaced by a third copy of leumorphin.

SEQ ID No. 4 signalpeptide of ppDyn
MAWQGLVLAA CLLMFPSTTA
SEQ ID No. 5 propeptide fragment of ppDyn without
known function
DCLSRCSLCA VKTQDGPKPI NPLICSLQCQ AALLPSEEWE
RCQSFLSFFT PSTLGLNDKE DLGSKSVGEG PYSELAKLSG
SFLKELEKSK FLPSISTKEN TLSKSLEEKL RGLSDGFREG
AESELMRDAQ LNDGAMETGT LYLAEEDPKE QV
SEQ ID No. 6 Neoendorphin
YGGFLRKYP
SEQ ID No. 7 Dyn A
YGGFLRRIRPKLKWDNQ
SEQ ID No. 8 Dyn B (rimorphin)
YGGFLRRQFKVVT
SEQ ID No. 9: Leumorphin
YGGFLRRQFKVVTRSQEDPNAYSGELFDA
SEQ ID No. 10 Dyn A modified
YGZFLRRZRPKLKWDNQ

Z stands for any amino acid, at least one is preferably substituted by another amino acid in comparison to the wild-type sequence.

SEQ ID No. 11 Dyn B modified
YGZFLRRZFKVVT

Z stands for any amino acid, at least one is preferably substituted by another amino acid in comparison to the wild-type sequence.

SEQ ID No. 12: Leumorphin modified
YGZFLRRZFKVVTRSQEDPNAYSGELFDA

Z stands for any amino acid, at least one is preferably substituted by another amino acid in comparison to the wild-type sequence.

SEQ ID No. 13 (ppDyn modified)
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLKELEKSK FLPSISTKEN
TLSKSLEEKL RGLSDGFREG AESELMRDAQ LNDGAMETGT
LYLAEEDPKE QVKRYGGFLR KYPKRSSEVA GEGDGDSMGH
EDLYKRYGZF LRRZRZKLKW DNQKRYGZ FLRRZFKVVT
RSQEDPNAYS GELFDA

Z stands for any amino acid, at least one is preferably substituted by another amino acid in comparison to the wild-type sequence.

SEQ Id No. 14 (Variant 1 of ppDyn modified)
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLKELEKSK FLPSISTKEN
TLSKSLEEKL RGLSDGFREG AESELMRDAQ LNDGAMETGT
LYLAEEDPKE QVKRYGZFLR RZFKVVTRSQ EDPNAYSGEL
FDAKRSSEVA GEGDGDSMGH EDLYKRYGZF LRRZRZKLKW
DNQKRYGZ FLRRZFKVVT RSQEDPNAYS GELFDA

Z stands for any amino acid, at least one is preferably substituted by another amino acid in comparison to the wild-type sequence.

SEQ ID No. 15 Variant 2 of ppDyn modified
MAWQGLVLAA CLLMFPSTTA DCLSRCSLCA VKTQDGPKPI
NPLICSLQCQ AALLPSEEWE RCQSFLSFFT PSTLGLNDKE
DLGSKSVGEG PYSELAKLSG SFLKELEKSK FLPSISTKEN
TLSKSLEEKL RGLSDGFREG AESELMRDAQ LNDGAMETGT
LYLAEEDPKE QVKRYGZFLR RZFKVVTRSQ EDPNAYSGEL
FDAKRSSEVA GEGDGDSMGH EDLYKRYGZF LRRZFKVVTR
SQEDPNAYSG ELFDAKRYGZ FLRRZFKVVT RSQEDPNAYS
GELFDA

Z stands for any amino acid, at least one is preferably substituted by another amino acid in comparison to the wild-type sequence.

### The Below Embodiments are Subject of the Present Invention

1. A delivery vector comprising a DNA sequence encoding pre-prodynorphyin or pre-prodynorphin-variants whereas said pre-prodynorphyin or pre-prodynorphin-variants comprise at least one of the following sequences selected from the group:
   a. Dyn A that is SEQ Id No. 7 (AA 207-223 of SEQ ID No. 1; ppDyn) or a variant thereof consisting of the first 13 AA (first from the N-terminal end) or a variant thereof consisting of the first 8 AA (first from the N-terminal end)
   b. Dyn B that is SEQ ID No. 8 (AA 226-238 of SEQ ID No. 1; ppDyn)
   c. leumorphin that is SEQ ID No. 9 (AA 226-254 of SEQ ID No. 1; ppDyn)
   d. variants of Dyn A according to SEQ Id No.7 having an amino acid sequence identity of at least 60% within the first 8 AA counted from the N-terminus of SEQ ID No. 7 (YGGFLRRI (SEQ ID NO: 18)).
   e. variants of Dyn B according to SEQ ID No. 8 having an amino acid sequence identity of at least 60% within the first 8 AA counted from the N-terminus of SEQ ID No. 8 (YGGFLRRQ (SEQ ID NO: 19)).
   f. variants of leumorphin according to SEQ ID No. 9 having an amino acid sequence identity of at least 60% within the first 8 AA counted from the N-terminus of SEQ ID No. 9 (YGGFLRRQ (SEQ ID NO: 19)).
2. A delivery vector according to embodiment 1, wherein the variants have an amino acid sequence identity of at least 70% within the first 8 AA counted from the N-terminus of SEQ ID No. 7 (YGGFLRRI (SEQ ID NO: 18)), SEQ ID No. 8 (YGGFLRRQ (SEQ ID NO: 19)) or SEQ ID No. 9 (YGGFLRRQ (SEQ ID NO: 19)), respectively.
3.
4. A delivery vector according to embodiment 1 or embodiment 2, wherein the variants have an amino acid sequence identity of at least 80% within the first 8 AA counted from the N-terminus of SEQ ID No. 7, SEQ ID No. 8 or SEQ ID No. 9, respectively.
5. A delivery vector according to any of embodiments 1 to 3 wherein said delivery vector comprises a DNA sequence encoding a pre-prodynorphin-variant that comprises at least one of the following sequences of variants selected from the group:

| SEQ ID No. 10 | |
|---|---|
| a. | (YGZFLRRZRPKLKWDNQ) |
| SEQ ID No. 11 | |
| b. | (YGZFLRRZFKVVT) |
| SEQ Id No. 12 | |
| c. | (YGZFLRRZFKVVTRSQEDPNAYSGELFDA) |

Z stands for any amino acid, at least one is preferably substituted
6. A delivery vector according to any of embodiments 1 to 4 wherein said delivery vector comprises multiple DNA sequences encoding SEQ ID No. 7, SEQ ID No. 8 and/or SEQ ID No. 9 or variants thereof wherein the sequences according to SEQ ID No. 7, SEQ ID No. 8 and/or SEQ ID No. 9 or variants thereof are flanked by peptidase recognition signals.
7. A delivery vector according to any of embodiments 1 to 5 wherein said delivery vector comprises multiple DNA sequences encoding SEQ ID No. 8 and/or SEQ ID No. 9 or variants thereof wherein the sequences according, SEQ ID No. 8 and/or SEQ ID No. 9 or variants thereof are flanked by peptidase recognition signals.
8. A delivery vector according to any of embodiments 1 to 6 wherein said delivery vector comprises multiple DNA sequences encoding SEQ ID No. 8 and/or SEQ ID No. 9 or variants thereof and wherein said delivery vector does not comprise DNA encoding SEQ ID No. 6 and/or 7.
9. A delivery vector according to any of embodiments 1 to 7 wherein said delivery vector comprises a DNA encoding SEQ ID No. 2 or SEQ ID No. 14.
10. A delivery vector according to any of embodiments 1 to 8 wherein said delivery vector comprises a DNA encoding SEQ ID No. 3 or SEQ ID No. 15.
11. A delivery vector according to any of embodiments 1 to 9 wherein said delivery vector comprises in addition a recombinant adeno-associated virus (AAV) vector genome or a recombinant lentivirus genome.
12. A delivery vector according to any of embodiments 1 to 10 comprising a recombinant AAV vector, wherein preferably said vector is a human or primate serotype vector.
13. A delivery vector according to any of embodiments 1 to 11 comprising a recombinant adeno-associated virus (AAV) vector genome, wherein said vector is a human serotype vector selected from the group comprising serotypes 1, 2, 3, 4, 5, 6, 7, 8, 9, 10, rh10, 11, 12, 13, 14, serpentine AAV, ancestral AAV or AAV capsid mutants derived thereof, preferably serotype 1 or 2.
14. A delivery vector according to any of the preceding embodiments, wherein said vector is a single stranded AAV vector or a self-complimentary (or dimeric) duplex vector.
15. A delivery vector according to any of the preceding embodiments, wherein the DNA sequence encoding pre-prodynorphyin or pre-prodynorphin-variants is operatively linked to expression control elements comprising a promoter and/or enhancer that produces sufficient expression of the gene product of interest to obtain a therapeutic effect, wherein the promoter and/or enhancer is selected from the group comprising constitutively active promoters e.g. CMV- or CBA promoter (chicken beta actin promoter and human cytomegalovirus IE gene enhancer), or inducible promoters comprising Gene Switch, tet-operon derived promotor, or neuron-specific promoters derived of e.g. phosphoglycerate kinase (PGK), synapsin-1 promoter (SYN), Neuron Specific Enolase (NSE).
16. A delivery vector according to any of the preceding embodiments, wherein said delivery vector further comprises a posttranscriptional regulatory element, preferably the woodchuck-hepatitis-virus-posttranscriptional-regulatory element.
17. A recombinant virus particle or a liposome, comprising a delivery vector according to any of the preceding embodiments.
18. The recombinant virus particle or liposome of embodiment 16, wherein said delivery vector comprises in addition a recombinant adeno-associated virus (AAV) vector genome and said rAAV vector genome is encapsidated in an AAV capsid or wherein said delivery vector comprises in addition a recombinant lentivirus vector genome and is packaged in a lentivirus particle.
19. A method of delivering a nucleic acid to a cell of the central nervous system, comprising contacting the cell with the delivery vector or recombinant virus particle or liposome of any of embodiments 1 to 17 under conditions sufficient for the DNA sequence encoding pre-prodynorphyin or pre-prodynorphin-variants to be introduced into the cell.
20. The method of embodiment 18, wherein the pre-prodynorphyin or pre-prodynorphin-variant is produced and released from the cell.
21. A method of delivering a nucleic acid to a cell of the central nervous system, comprising contacting the cell with the recombinant virus particle or liposome of embodiment 18 or 19 under conditions sufficient for the DNA sequence encoding pre-prodynorphyin or pre-prodynorphin-variants to be introduced into the cell.
22. The method of embodiment 20, wherein the pre-prodynorphyin or pre-prodynorphin-variant is produced and released from the cell.
23. A delivery vector or recombinant virus particle or liposome according to any of embodiments 1 to 17 for use as medicament.
24. A delivery vector or recombinant virus particle or liposome according to any of embodiments 1 to 17 for use in treating focal epilepsy in a subject, in particular mesial temporal lobe epilepsy.
25. A delivery vector or recombinant virus particle or liposome according to any of embodiments 1 to 17 in treating focal epilepsy, in particular mesial temporal lobe epilepsy, in a subject, wherein said vector is suitable for transduction of neuronal cells of the brain.
26. A delivery vector or recombinant virus particle or a liposome according to any of embodiments 1 to 17 for use in treating focal epilepsy in a subject, wherein said vector or recombinant virus particle is suitable for peripheral administration or for intracranial or for intracerebral or for intrathecal administration.
27. A pharmaceutical composition comprising a delivery vector or recombinant virus particle according to any of embodiments 1 to 17, and optionally a pharmaceutically acceptable carrier.
28. A cell infected, preferably in vitro or ex vivo, with a delivery vector or recombinant virus or liposome particle according to embodiments 1 to 17.
29. A method of treating a subject with focal epilepsy comprising administering a delivery vector, a recombinant virus particle, or a pharmaceutical composition as defined in embodiments 1 to 17 and 26 to the subject.
30. A method according to embodiment 28, wherein said method comprises any of the methods as defined in embodiments 18 to 21.
31. Peptides with agonistic effects on human Kappa Opiod Receptors (KOR) derived from any of the delivery vectors according to embodiments 1-15.
32. Peptides with agonistic effects on human Kappa Opiod Receptors (KOR) selected from the group comprising peptides of SEQ ID No. 6, SEQ ID No. 7, SEQ ID No. 8, SEQ ID No. 9, SEQ ID No. 10, SEQ ID No. 11, and SEQ ID No. 12.

### Further Embodiments are Subject of the Present Invention

1. A delivery vector comprising a DNA sequence encoding pre-prodynorphyin or pre-prodynorphin-variants and wherein said delivery vector drives expression of a pre-propeptide that is pre-prodynorphyin or a pre-prodynorphin-variant wherein said pre-propeptides comprise a signalpeptide and, whereas said pre-prodynorphyin or pre-prodynorphin-variants comprise at least one of the following sequences selected from the group:
   a. Dyn A that is SEQ Id No. 7 (AA 207-223 of SEQ ID No. 1; ppDyn) or a variant thereof consisting of the first 13 AA (first from the N-terminal end) or a variant thereof consisting of the first 8 AA (first from the N-terminal end)
   b. Dyn B that is SEQ ID No. 8 (AA 226-238 of SEQ ID No. 1; ppDyn)
   c. leumorphin that is SEQ ID No. 9 (AA 226-254 of SEQ ID No. 1; ppDyn)
   d. variants of Dyn A according to SEQ Id No.7 having an amino acid sequence identity of at least 60% within the first 8 AA counted from the N-terminus of SEQ ID No. 7 (YGGFLRRI (SEQ ID NO: 18)) i.e. having an amino acid sequence identity of at least 60% within the sequence YGGFLRRI (SEQ ID NO: 18) comprised in SEQ ID No. 7.
   e. variants of Dyn B according to SEQ ID No. 8 having an amino acid sequence identity of at least 60% within the first 8 AA counted from the N-terminus of SEQ ID No. 8 (YGGFLRRQ (SEQ ID NO: 19)) i.e. having an amino acid sequence identity of at least 60% within the sequence YGGFLRRQ (SEQ ID NO: 19) comprised in SEQ ID No. 8.
   f. variants of leumorphin according to SEQ ID No. 9 having an amino acid sequence identity of at least 60% within the first 8 AA counted from the N-terminus of SEQ ID No. 9 (YGGFLRRQ (SEQ ID NO: 19)), i.e. having an amino acid sequence identity of at least 60% within the sequence YGGFLRRQ (SEQ ID NO: 19) comprised in SEQ ID No. 9.
2. A delivery vector according to embodiment 1, wherein the variants have an amino acid sequence identity of at least 70% within the first 8 AA counted from the N-terminus of SEQ ID No. 7 (YGGFLRRI (SEQ ID NO: 18)), SEQ ID No. 8 (YGGFLRRQ (SEQ ID NO: 19)) or SEQ ID No. 9 (YGGFLRRQ (SEQ ID NO: 19)), respectively.
3. A delivery vector according to embodiment 1 or embodiment 2, wherein the variants have an amino acid sequence identity of at least 80% within the first 8 AA counted from the N-terminus of SEQ ID No. 7, SEQ ID No. 8 or SEQ ID No. 9, respectively.
4. A delivery vector according to any of embodiments 1 to 3 and wherein said delivery vector drives expression of a pre-propeptide that is pre-prodynorphyin or a pre-prodynorphin-variant wherein said pre-propeptide comprise a signalpeptide and, wherein said delivery vector comprises a DNA sequence encoding a pre-prodynorphin-variant that comprises at least one of the following sequences of variants selected from the group:

| SEQ ID No. 10 | |
|---|---|
| a. | (YGZFLRRZRPKLKWDNQ) |
| SEQ ID No. 11 | |
| b. | (YGZFLRRZFKVVT) |
| SEQ Id No. 12 | |
| c. | (YGZFLRRZFKVVTRSQEDPNAYSGELFDA), |

wherein Z stands for any amino acid, and wherein at least one Z in a sequence according to a.; b. or c. is preferably substituted by another amino acid when compared to the wild-type sequence of said dynorphin fragment according to a sequence according to a.; b. or c.
5. A delivery vector according to any of embodiments 1 to 4 wherein said delivery vector comprises in addition a recombinant adeno-associated virus (AAV) vector genome or a recombinant lentivirus genome.
6. A delivery vector according to any of embodiments 1 to 5 comprising a recombinant adeno-associated virus (AAV) vector genome, wherein said vector is a human serotype vector selected from the group comprising serotypes 1, 2, 3, 4, 5, 6, 7, 8, 9, 10, rh10, 11, 12, 13, 14, serpentine AAV, ancestral AAV or AAV capsid mutants derived thereof, preferably serotype 1 or 2.
7. A recombinant virus particle or a liposome, comprising a delivery vector according to any of the preceding embodiments.
8. The recombinant virus particle or liposome of embodiment 7, wherein said delivery vector comprises in addition a recombinant adeno-associated virus (AAV) vector genome and said rAAV vector genome is encapsidated in an AAV capsid or wherein said delivery vector comprises in addition a recombinant lentivirus vector genome and is packaged in a lentivirus particle.
9. A method of delivering a nucleic acid to a cell of the central nervous system, comprising contacting the cell with the delivery vector or recombinant virus particle or liposome of any of embodiments 1 to 8 under conditions sufficient for the DNA sequence encoding pre-prodynorphyin or pre-prodynorphin-variants to be introduced into the cell.
10. A delivery vector or recombinant virus particle or liposome according to any of embodiments 1 to 8 for use as medicament.
11. A delivery vector or recombinant virus particle or liposome according to any of embodiments 1 to 8 for use in treating focal epilepsy in a subject, in particular mesial temporal lobe epilepsy, or for use in preventing epileptic seizures in a subject that suffers from focal epilepsy whereby said delivery vector or recombinant virus particle or liposome provides activation of human Kappa Opiod Receptors in the epileptogenic focus, thereby inhibiting seizures.
12. A delivery vector or recombinant virus particle or liposome according to any of embodiments 1 to 8 for use in treating focal epilepsy in a subject, in particular mesial temporal lobe epilepsy, or for use in preventing epileptic seizures in a subject that suffers from focal epilepsy whereby said delivery vector or recombinant virus particle or liposome provides activation of human Kappa Opiod Receptors in the epileptogenic focus, thereby inhibiting seizures whereby said delivery vector or recombinant virus particle or liposome leads to on-demand release of peptides with agonistic effects on human Kappa Opiod Receptors in the epileptogenic focus.
13. A delivery vector or recombinant virus particle or a liposome according to any of embodiments 1 to 8 for use in treating focal epilepsy in a subject, in particular mesial temporal lobe epilepsy, or for use in preventing epileptic seizures in a subject that suffers from focal epilepsy according to embodiments 10-12, wherein said vector or recombinant virus particle is suitable for peripheral administration or for intracranial or for intracerebral or for intrathecal or for intraparenchymal administration.
14. A delivery vector or recombinant virus particle or a liposome according to any of embodiments 1 to 8 for use in treating focal epilepsy in a subject, in particular mesial temporal lobe epilepsy, or for use in preventing epileptic seizures in a subject that suffers from focal epilepsy according to embodiments 10-13, wherein said delivery vector or recombinant virus particle or a liposome is applied intracerebral, preferred is applied focal.
15. A pharmaceutical release-on-demand composition delivery vector or recombinant virus particle or liposome according to any of embodiments 1 to 8, and optionally a pharmaceutically acceptable carrier.
16. A cell infected, preferably in vitro or ex vivo, with a delivery vector or recombinant virus or liposome particle according to embodiments 1 to 8.
17. A method of treating a subject with focal epilepsy in particular mesial temporal lobe epilepsy, or a method of preventing epileptic seizures in a subject that suffers from focal epilepsy:
   comprising administering a delivery vector, a recombinant virus particle, or a pharmaceutical composition as defined in embodiments 1 to 8 to the subject, whereby preferably said delivery vector or recombinant virus particle or liposome encode pre-propeptides, which after maturation and release provide activation of human Kappa Opiod Receptors in the epileptogenic focus, thereby inhibiting seizures, amd wherein preferably said delivery vector or recombinant virus particle or a liposome is applied intracerebral, preferably applied focal.
18. Peptide with agonistic effects on human Kappa Opiod Receptors (KOR) derived from any of the delivery vectors according to embodiments 1-6, wherein preferably said peptide is selected from the group comprising the peptides having SEQ ID No.s 10, SEQ ID No.s 11, SEQ ID No.s 12, SEQ ID No.s 13, SEQ ID No.s 14 and SEQ ID No.s 15.
19. Peptide with agonistic effects on human Kappa Opiod Receptors (KOR) wherein said peptide is selected from the group comprising the peptides having SEQ ID No.s 10, SEQ ID No.s 11, SEQ ID No.s 12, SEQ ID No.s 13, SEQ ID No.s 14 and SEQ ID No.s 15.
20. A pharmaceutical release-on-demand composition comprising a peptide according to embodiments 18 or 19.

### FIGURE DESCRIPTION

FIG. 1

Depicted is an AAV vector backbone including all regulatory genetic elements to express human pre-prodynorphin (red) or IGFP (blue).

FIG. 2

EEG recordings obtained from the ipsi-lateral hippocampus of a kainic acid injected animal from two days before to seven days after rAAV-pDyn (SEQ ID No. 1) injection.

FIG. 3

Frequency of generalized seizures obtained by EEG recordings from the ipsi-lateral hippocampus and motor cortex of kainic acid injected animals from two days before to 4 months after rAAV-pDyn injection. pDyn=SEQ ID No. 1; Var. 1=SEQ ID No. 2; Var. 2=SEQ ID No. 3

FIG. 4

Amount of processed Dyn B (Dyn B=SEQ ID No. 8) released during different types of stimulation measured by micro-dialysis and subsequent EIA. pDyn knockout mice were injected with rAAV pDyn (pDyn=SEQ ID No. 1; Var. 2=SEQ ID No. 3) 3 weeks before the experiment. As these mice do not express endogenous pDyn, all Dyn B measured is vector-derived.

FIG. 5 (A-F)

Barnes maze probe tests from different groups of naïve or KA injected animals treated with rAAV. For the memory test the maze is divided in quarters. The Q1 is the one containing the target hole, Q2 and Q4 are the quarters surrounding the Q1 and Q3 is the opposite one. Animals (except those depicted in C) were injected with rAAV either expressing □GFP or pDyn Seq ID no. 1. In A and B, data for mice injected with rAAV, but not treated with kainic acid are depicted. Panel C shows entirely untreated animals. Panels D, E and F show the same set of animals treated with rAAV two weeks after kainic acid at time intervals after kainic acid as stated in the panel.

FIG. 6

Pentylenetetrazole is an inhibitor of GABAA receptors and induces seizures upon injection into the tail vein. pDyn deficient animals display a reduce seizure threshold compared to wild-type mice. This can be rescued by treatment with Dyn B. Dyn B variants (Dyn B=SEQ ID No. 8, Dyn B G3A=SEQ ID No. 11 wheren Z at position 3 is alanine, Dyn B G3A+Q8A=SEQ ID No. 11 wheren Z at position 3 is alanine and Z at position 8 of SEQ ID No. 11 is alanine, Dyn B Q8A=SEQ ID No. 11 wheren Z at position 8 is alanine) with a replacement of glycin in position 3 by alanin show higher potency in this test, suggesting that a lower concentration of these peptides can elicit anticonvulsant effects. From this, we expect an early onset of the effect of gene therapy and a lower number of vectors needed per patient.

### REFERENCES

Ausubel, F. M. et al. (eds.), Current Protocols in Molecular Biology, Greene Publishing Associates, Wiley (1989)
Berns K. I. and C. R. Parrish in FIELDS VIROLOGY, eds. D. N. Knipe and P. M. Howley, volume 2, chapter 57 (6th ed., 2013, Wolters-Kluwer, Lippincott Williams & Wilkins Publishers).
Coatsworth J J. Studies on the clinical effect of marketed antiepileptic drugs. 1971. NINDS Monograph 12
de Lanerolle N C, Williamson A, Meredith C, Kim J H, Tabuteau H, Spencer D D, Brines M L. Dynorphin and the kappa 1 ligand [3h]u69,593 binding in the human epileptogenic hippocampus. 1997. Epilepsy Res 28:189-205.
Engel J J. Mesial temporal lobe epilepsy: What have we learned? 2001. Neuroscientist 7:340-352.
Gambardella A, Manna I, Labate A, Chifari R, Serra P, La Russa A, LePiane E, Cittadella R, Andreoli V, Sasanelli F, Zappia M, Aguglia U, Quattrone A. Prodynorphin gene promoter polymorphism and temporal lobe epilepsy. 2003. Epilepsia 44:1255-1256.
Gossen M and Bujard H, (1992) Tight control of gene expression in mammalian cells by tetracyclin-responsive promoters. Proc. Natl. Acad. Sci. USA 89:5547-51.
Gossen M, Freundlieb S, Bender G, Müller G, Hillen W and Bujard H, (1995) Transcriptional activation by tetracyclines in mammalian cells. Science 268 (5218): 1766-9.
Grimm D, Kay M A, Kleinschmidt J A (2003) Helper virus-free, optically controllable, and two-plasmid-based production of adeno-associated virus vectors of serotypes 1 to 6, Mol Ther 7 (6) 839-50
Harvey D M, Caskey C T (1998) Inducible control of gene expression: prospects for gene therapy. Curr. Opin. Chem. Biol. 2:512-8.
Henriksen S J, Chouvet G, McGinty J, Bloom F E. Opioid peptides in the hippocampus: Anatomical and physiological considerations. 1982. Ann N Y Acad Sci 398:207-220.
Hüser D, Gogol-Döring A, Chen W, Heilbronn R (2014) Adeno-associated virus type 2 wild-type and vector-mediated genomic integration profiles in human diploid fibroblasts analyzed by 3^{rd} generation PacBio DNA sequencing. J Virol 188 (19): 11253-11263
Loacker S, Sayyah M, Wittmann W, Herzog H, Schwarzer C. Endogenous dynorphin in epileptogenesis and epilepsy: Anticonvulsant net effect via kappa opioid receptors. 2007. Brain 130:1017-1028.
Loftus S K, Erickson R P, Walkley S U, Bryant M A, Incao A, Heidenreich R A, Pavan W J (2002), Rescue of neurodegeneration in Niemann-Pick C mice by a prion promoter-driven Npc1 cDNA transgene. Hum. Mol. Genet. 11:3107-14.
Loscher W, Schmidt D. Modern antiepileptic drug development has failed to deliver: Ways out of the current dilemma. 2011. Epilepsia 52:657-678.
Magari S R Rivera V M, Iulicci J D, Gilman M, Cerasoli F Jr, (1997) Pharmacologic control of a humanized gene therapy system implanted into nude mice J. Clin. Invest. 100:2865-72
McCarty D M, Monahan P E, Samulski R J (2001) Self-complementary recombinant adeno-associated virus (scAAV) vectors promote efficient transduction independently of DNA synthesis, Gene Ther 8 (16) 1248-54
McNamara J O. Emerging insights into the genesis of epilepsy. 1999. Nature 399: A15-22.
Mietzsch M, Broecker F, Reinhardt A, Seeberger P H, Heilbronn R (2014) Differential adeno-associated virus serotype-specific interaction patterns with synthetic heparins and other glycans. J Virol 88:2991-3003
Mietzsch M, Grasse S, Zurawski C, Weger S, Bennett A, Agbandje-McKenna M, Muzyczka N, Zolotukhin S, Heilbronn R (2014) OneBac: Platform for scalable and high-titer production of adeno-associated virus serotype 1-12 vectors for gene therapy. Hum Gene Ther 25:212-222
Muzyczka (1992) Use of adeno-associated virus as a general transduction vector for mammalian cells. Curr. Topics Microbiol. Immunol. 158:97-129).
No D, Yao T P Evans R M (1996) Ecdysone-inducible gene expression in mammalian cells and transgenic mice Proc. Natl. Acad. Sci. USA 93:3346.-51
Pillay S, Meyer N L, Puschnik A S, Davulcu O, Diep J, Ishikawa Y, Jae L T, Wosen J E, Nagamine C M, Chapman M S, Carette J E (2016) Nature 530 (7588) 108-12.
Pirker S, Gasser E, Czech T, Baumgartner C, Schuh E, Feucht M, Novak K, Zimprich F, Sperk G. Dynamic up-regulation of prodynorphin transcription in temporal lobe epilepsy. 2009. Hippocampus 19:1051-1054.
Rosenzweig A (2007), Vectors for Gene Therapy. In: Current Protocols in Human Genetics. Wiley John and Sons, Inc.: DOI: 10.1002/0471142905.hg1200s52.
Schunk E, Aigner C, Stefanova N, Wenning G, Herzog H, Schwarzer C. Kappa opioid receptor activation blocks progressive neurodegeneration after kainic acid injection. 2011. Hippocampus 21:1010-1020.
Schwarzer C. 30 years of dynorphins—new insights on their functions in neuropsychiatric diseases. 2009. Pharmacol Ther 123:353-370.
Siggins G R, Henriksen S J, Chavkin C, Gruol D. Opioid peptides and epileptogenesis in the limbic system: Cellular mechanisms. 1986. Adv Neurol 44:501-512.
Simonato M, Romualdi P. Dynorphin and epilepsy. 1996. Prog Neurobiol 50:557-583.
Solbrig M V, Adrian R, Chang D Y, Perng G C. Viral risk factor for seizures: Pathobiology of dynorphin in herpes simplex viral (hsv-1) seizures in an animal model. 2006. Neurobiol Dis 23:612-620.
Spencer S, Huh L. Outcomes of epilepsy surgery in adults and children. 2008. Lancet Neurol 7:525-537.
Stogmann E, Zimprich A, Baumgartner C, Aull-Watschinger S, Hollt V, Zimprich F. A functional polymorphism in the prodynorphin gene promotor is associated with temporal lobe epilepsy. 2002. Ann Neurol 51:260-263.
Stutika C, Gogol-Doring A, Botschen L, Mietzsch M, Weger S, Feldkamp M, Chen W, Heilbronn R (2015) A comprehensive RNA-Seq analysis of the adeno-associated virus type 2 transcriptome reveals novel AAV transcripts, splice variants, and derived proteins. J Virol 90 (3) 1278-89
Takahashi M, Senda T, Tokuyama S, Kaneto H. Further evidence for the implication of a kappa-opioid receptor mechanism in the production of psychological stress-induced analgesia. 1990. Jpn J Pharmacol 53:487-494.
Tani M, Fuentes M E, Petersen J W, Trapp B D, Durham S K, Loy J K, Bravo R, Ransohoff R M, Lira S A (1996) Neutrophil infiltration, glial reaction, and neurological disease in transgenic mice expressing the chemokine N51/KC in oligodendrocytes. J. Clin. Invest. 98:529-39.
Toll, L., Berzetei-Gurske, I. P., Polgar, W. E., Brandt, S. R., Adapa, I. D., Rodriguez, L., et al. (1998). Standard binding and functional assays related to medications development division testing for potential cocaine and opiate narcotic treatment medications. NIDA Res Monogr 178, 440-466.
Tortella F C. Endogenous opioid peptides and epilepsy: Quieting the seizing brain? 1988. Trends Pharmacol Sci 9:366-372.
Wang Y, DeMayo F J, Tsai S Y, O'Malley B W (1997) Ligand-inducible and liver-specific target gene expression in transgenic mice. Nat. Biotech. 15:239-43
Wang Y, Xu J, Pierson T, O'Malley B W, Tsai S Y (1997) Positive and negative regulation of gene expression in eucaryotic cells with an inducible transcriptional regulator. Gene Ther, 4:432-41.
Xiao X, Li J, Samulski R J (1998) Production of high-titer recombinant adeno-associated virus vectors in the absence of helper adenovirus, JVirol 72 (3) 2224-32
Zangrandi L, Burtscher J, MacKay J, Colmers W, & *Schwarzer C* (2016) The G-protein biased partial kappa opioid receptor agonist 6′-GNTI blocks hippocampal paroxysmal discharges without inducing aversion. British J Pharmacol, 173 (11): 1756-67,doi: 10.1111/bph.13475

### EXAMPLES

### Example 1

### 1.1 AAV Constructs

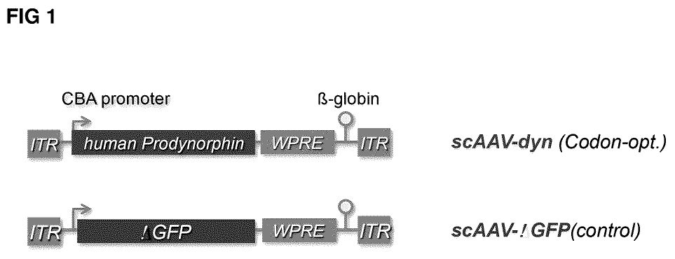

Plasmids with the AAV2-ITR-flanked vector backbones contained the human CMV-IE gene enhancer followed by a truncated version of the chicken beta actin promoter. These drive expression of the full-length codon-optimized human ppdyn sequence (AA SEQ ID No. 1) or either of the variants (AA SEQ ID No. 2; SEQ ID No. 3) or a truncated variant (IGFP) or the full length form of EGFP. Gene expression was enhanced by the woodchuck hepatitis virus posttranslational enhancer element (WPRE) followed by a poly A⁺ signal sequence derived from bovine growth hormone. The AAV2 ITR was either in its wildtype configuration to yield AAV vectors with ssDNA genomes, or one ITR was truncated so that self-complementary AAV vectors with dsDNA genomes resulted (FIG. 1).

### 1.2 AAV Vector Preparation

HEK 293 cells were seeded at 25-33% confluency in DMEM with 5% FCS. Cells were transfected 24 hr later by calcium phosphate cotransfection. AAV vectors were produced essentially as described elsewhere (Mietzsch, Grasse et al., 2014). In brief: The two-plasmid (pDG) cotransfection protocol using plasmids for AAV rep, cap, Ad5 helper genes, and the plasmid for rAAV expressing ppdyn (SEQ ID No. 1) or variants thereof (SEQ ID No. 2; SEQ ID No. 3) as described above. The medium was replaced 12 hr later by medium with reduced FCS content (2%). Cultures were harvested 72 hr after transfection by three freeze-thaw cycles for cell lysis. Crude lysates were digested with 250U/ml benzonase (Merck) at 37° C. for 1 hr to degrade input and unpackaged plasmid DNA, centrifuged at 8,000 g for 30 min to pellet the cell debris.

1.3 rAAV Purification

rAAV vectors were packaged in serotypes 1 or 2 capsids and were purified from benzonase treated, cleared freeze-thaw supernatants by one-step heparin sepharose chromatography or by AVB sepharose affinity chromatography using 1 ml prepacked HiTrap columns on an ÄKTA purifier (GE Healthcare) as follows: Freeze-thaw supernatants were diluted 1:1 in 1. phosphate-buffered saline (PBS) supplemented with 1 mM MgCl2 and 2.5 mM KCl (1. PBSMK) before loading on the column at 0.5 ml/min. The column was washed with 20 ml of 1. PBS-MK at a rate of 1 ml/min. AAV vectors were eluted with 0.1 M sodium acetate and 0.5 M NaCl pH 2.5 at a rate of 1 ml/min and neutralized immediately with 1/10 volume of 1 M Tris-HCl pH 10. Purified rAAV preparations were dialyzed against 1×PBS-MK using Slide-A-Lyzer dialysis cassettes (10,000 MWCO; Thermo Scientific) (Mietzsch, Grasse et al., 2014).

1.4. Quantification of rAAV Vector Preparations

Highly purified rAAV vector preparations were digested with Proteinase K for 2 hr at 56° C. DNA was purified by repeated extractions with phenol and chloroform and precipitated with ethanol. Serial dilutions of capsid-released AAV genomes were analyzed by quantitative Light-Cycler PCR, using the Fast Start DNA Master SYBR Green kit (Roche). PCR primers were specific for the bovine growth hormone gene-derived polyA site of the vector backbone (5′-CTAGAGCTCGCTGATCAGCC-3′ (SEQ ID NO: 16) and 5′-TGTCTTCCCAATCCTCCCCC-3′ (SEQ ID NO: 17)). The titer of the highly purified AAV preparations was measured as AAV-DNA containing genomic particles (gp)/ml (Mietzsch, Grasse et al., 2014).

### Example 2

### Animals

C57BL/6N wild-type and pDyn knockout (pDyn-KO) mice were investigated in this study. pDyn-KO mice were backcrossed onto the C57BL/6N background over 10 generations (Loacker et al., 2007). For breeding and maintenance, mice were group-housed with free access to food and water. Temperature was fixed at 23° C. and 60% humidity with a 12 h light-dark cycle (lights on 7 am to 7 pm). All procedures involving animals were approved by the Austrian Animal Experimentation Ethics Board in compliance with the European Convention for the Protection of Vertebrate Animals Used for Experimental and Other Scientific Purposes ETS no.: 123, and the Canadian Council on Animal Care. Every effort was taken to minimise the number of animals used.

### Example 3

### Kainic Acid Injection and Electrodes Implantation

Male mice (12-14 weeks) were sedated with ketamine (160 mg/kg, i.p.; Graeub Veterinary Products, Switzerland) and then deeply anesthetised with sevoflurane through a precise vaporizer (Midmark, USA). Mice were injected with 50 nl of a 20 mM KA solution into the hippocampus (RC −1.80 mm; ML +1.80 mm; DV −1.60 mm) as previously described (Loacker et al., 2007). Two electrodes (one cortical and one depth electrodes) were implanted immediately after KA administration. Epoxylite-coated tungsten depth electrodes (diameter 250 μm; FHC, USA) were placed into the hippocampus aimed at the CA1 area (RC −1.80 mm; ML +1.80 mm; DV −1.60 mm). Surface electrodes were gold-plated screws placed into the skull on top of the motor-cortex (RC +1.70 mm; ML +1.6 mm with the bregma as a reference point) to monitor the generalisation of abnormal EEG activities. An additional surface electrode was placed on the cerebellum as ground and reference. Electrodes were secured in place with dental acrylate (Heraeus Kulzer GmbH, Germany).

### Example 4

rAAV Injections

For experiments requiring rAAV administration, a guide cannula was implanted, which was attached to the hippocampal depth electrode. All animals received meloxicam (2 mg/kg) 20 minutes before surgery as an analgesic treatment. For the rAAVs injections animals were mildly anesthetized in a sevoflurane chamber during the time of the injection (20 minutes). The injection was made through the guide cannula with an injection pump at a flow of 0.1 L/min, a total volume of 2 μL was injected.

### Example 5

### EEG Recoding and Analysis

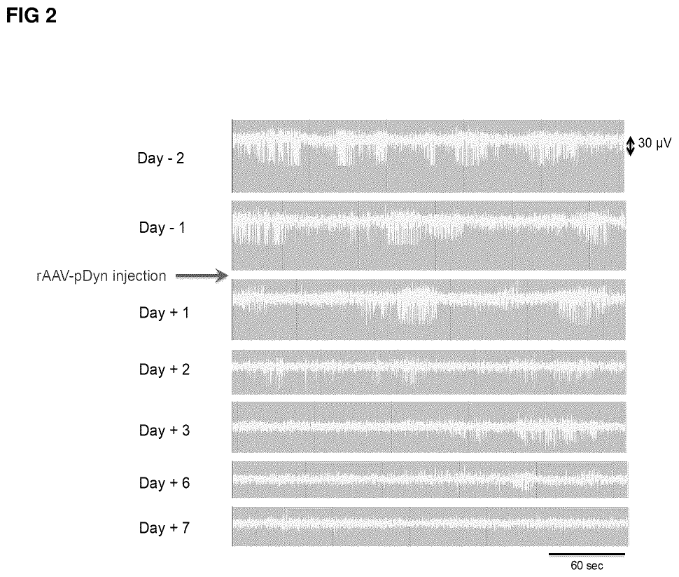

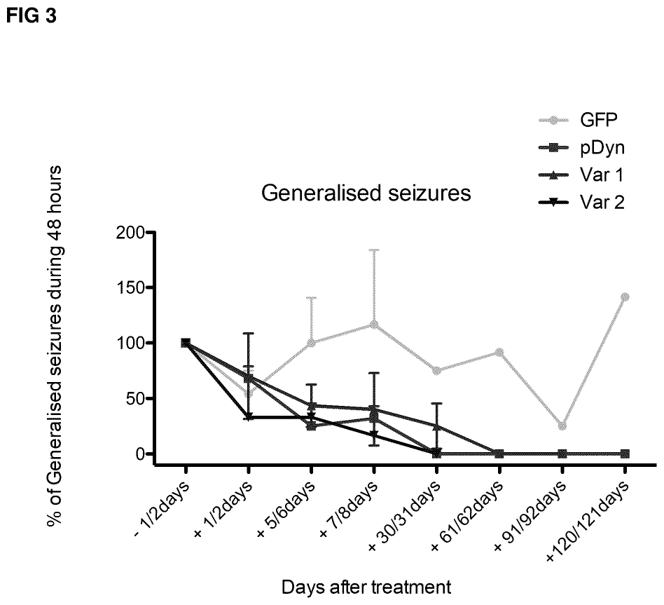

The EEG was obtained using a wireless recording device (Neurologger, TSE, Germany) and automatically analysed using SciWorks Software (Datawave Technologies, USA). EEGs were filtered for epileptiform spikes defined as a high amplitude discharges (>3× baseline) lasting less than 70 ms. Spike trains were defined as the occurrence of at least three spikes with a frequency higher than 1 Hz and lasting for at least 1 s (see FIG. 2). Spikes with lower frequencies were counted as inter-ictal spikes. Prolonged hippocampal paroxysmal discharges (hpd) were defined as spike trains lasting for a minimum of 10 sec (Zangrandi et al., 2016). Generalized seizures were assessed visually by co-appearance for high voltage EEG abnormalities in the hippocampal depth and motor-cortical surface electrode (see FIG. 3).

### Example 6

### Spatial Memory Testing (Barnes Maze)

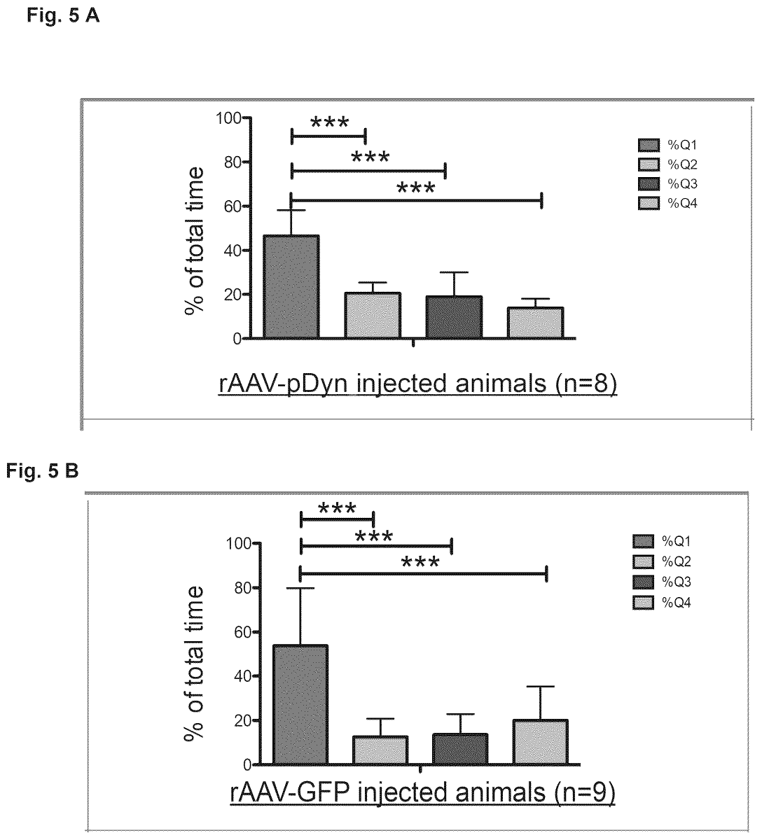

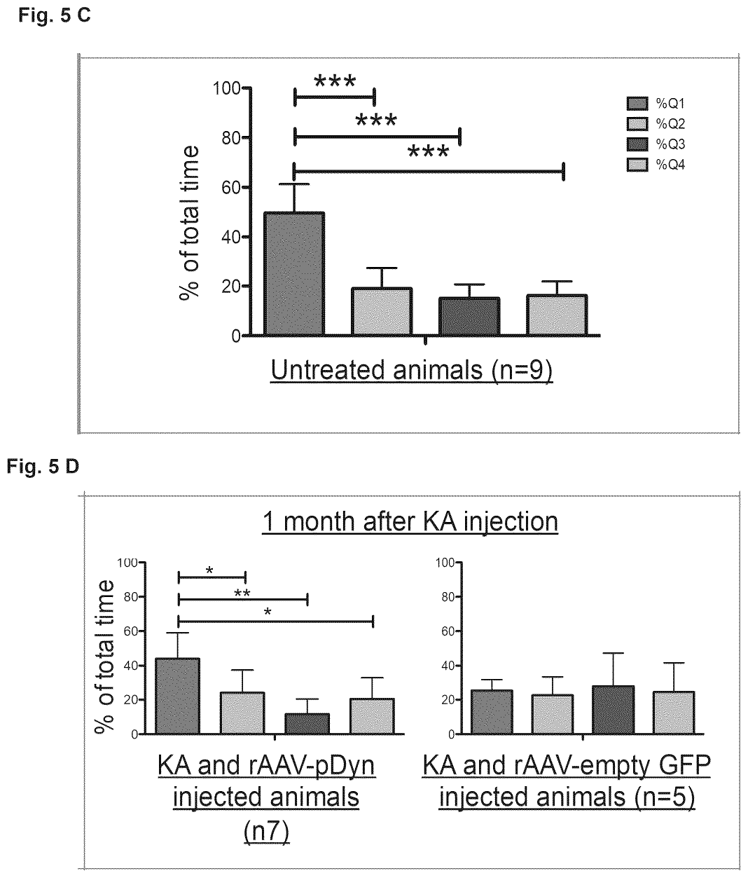

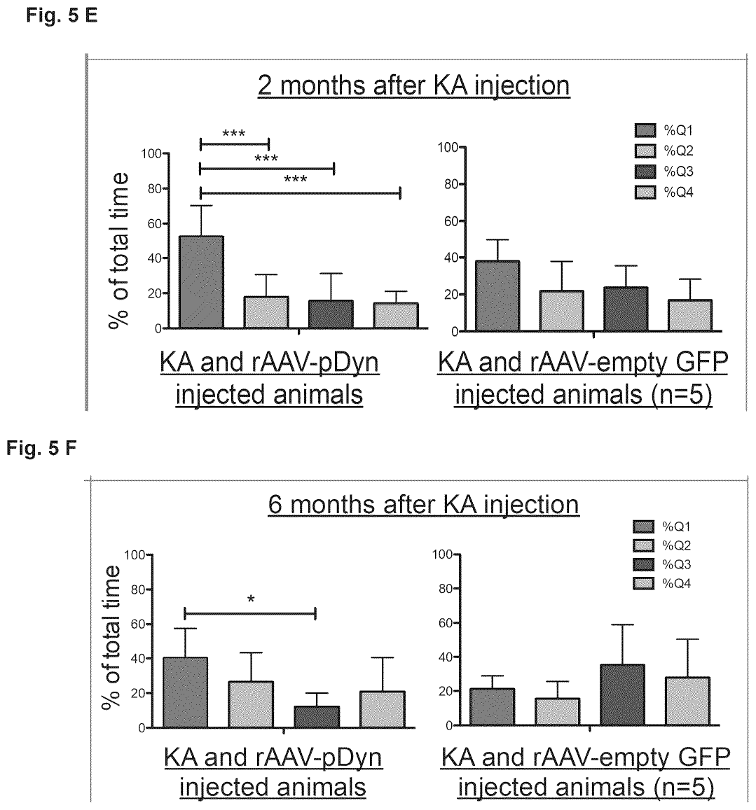

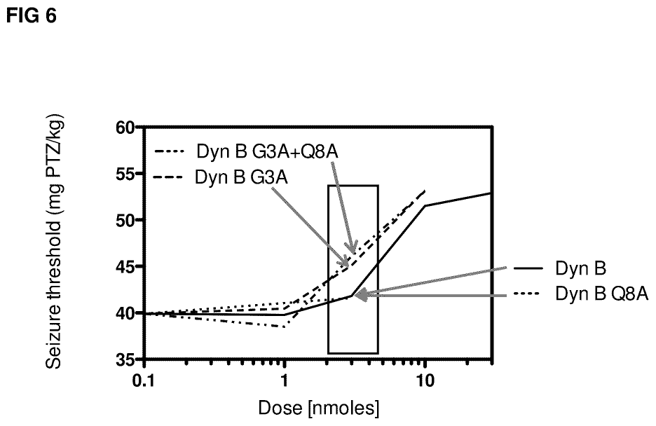

To assess learning and memory of naive and treated animals the Barnes maze was used. the Barnes maze was executed at 60 lux on a flat circular table (diameter 100 cm) with 20 holes around its perimeter. Amongst those, only one allowed the mouse to exit the maze into an escape dark box. Visual clues were placed around the disk with an interval of 90°. The first day was the habituation day, mice were allowed to freely explore the maze during 5 minutes with the target hole open. The position of the escape box was kept constant during the entire experiment. When the mouse found the hole, the box was closed and the animal was kept in there for 2 minutes to let it associate the escape box as a secure place. If the animal didn't find the target hole during the 5 minutes, the animal was gently guided to the hole. Acquisition was made during the next 4 days, 3 trials of 3 minutes maximum were performed, as soon as the mouse find the hole, it was kept for 2 minutes inside. If the mice didn't find it after the 3 minutes it was gently guide to the hole. The primary errors done by the animal and the latency to find the hole were calculated and defined as learning criteria. The probe trial was executed one the sixth day. All the holes were closed and the mouse was free to explore the maze during 5 minutes. For evaluation, the board was divided in quadrants and the time spent in each quadrant was measured (see FIG. 5). The quadrant previously containing the open escape hole is referred to as Q1, this is flanked by Q2 and Q4, while Q3 is opposing Q1.

### Example 7

### Microdialysis

Microdialysis was performed on pDyn-KO animals which had received rAAV-pDyn injection into one hippocampus as described above 3 weeks before the microdialysis experiment. At the time of vector injection (RC −1.80 mm; ML +1.80 mm; DV −1.60 mm), animals were implanted with a stimulation electrode (RC −4.20 mm; ML +3.20 mm; DV −4.90 mm) and a guide cannula targeting the hilus of the rAAV-injected hippocampus. For microdialysis MAB-2 probes (SciPro, Sanborn, NY) were placed into the hippocampus and flushed by artificial CSF (140 mM NaCl; 3.0 mM KCl; 1.25 mM CaCl₂); 1.0 mM MgCl₂; 1.2 mM Na₂HPO₄; 0.3 mM NaH₂PO₄; 3 mM glucose, pH 7.2) at a rate of 0.4 μL/min. ACSF was collected for 3×25 min followed by 25 min low frequency stimulation (300 μA; isolated 0.3 msec square pulses with 10 sec interval, ISO-STIM 01D, NPI, Tamm, Germany). After another 25 min baseline, 25 min of high frequency stimulation were performed (150 μA; trains of 0.3 msec square pulses 20 msec apart for 1 sec; trains were separated by 10 sec.).

### Example 8

### Dynorphin B Enzyme Immunoassay (EIA)

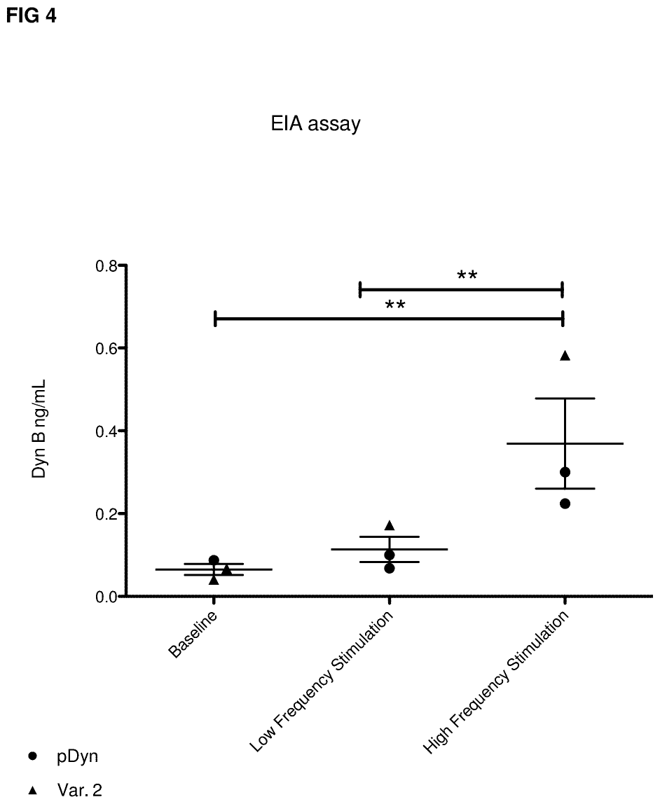

The content of processed Dynorphin B in the eluate of microdialysis experiments was measured by a specific Dyn B (SEQ ID No. 8) EIA (S-1429; Peninsula; San Carlos, CA), according to manufacturer's manual. In short, samples were incubated with the antiserum for 1 hour, followed by an overnight incubation with the Bt-tracer. On the second day, streptavidin-HRP was added for one hour after five washes with EIA buffer. After another 5 washes, samples were reacted with TMB solution for 5 minutes and then analysed on a plate reader a 450 nm. Dyn B (SEQ ID No. 8) content was analysed based on calibration samples run in parallel and expressed as ng/ml (see FIG. 4).

### Example 9

### Statistical Analysis

Following acquisition, electrophysiological recordings were viewed and analysed using pClamp 10.3 (Molecular devices). Prism 5 for Mac (version 5.0f) was used to perform a statistical analysis of in vivo experiments and to generate figures. For the statistical analysis, a one-way ANOVA with a Dunnett post hoc test was applied to in vivo experiments. For electrophysiology, the two-tailed, paired t-tests were applied for resting membrane potential analysis, and a one-way ANOVA was used to compare drug effects on IPSC. A p value less than 0.05 was considered significant. Data are presented as mean±standard error of the mean (SEM).

### Example 10

### Seizure Threshold

The seizure threshold is a measure for the susceptibility to develop seizures. The resistance against seizure-inducing agents or stimuli is used as readout in animals. Infusion of pentylenetetrazole, a GABAA receptor antagonist, into the tail vein of rodents is an accepted method to measure the seizure threshold. Anticonvulsant activity of substances or treatments can be demonstrated by an increased seizure threshold upon application. pDyn deficient mice display a lower seizure threshold than wild-type mice. This can be rescued by kappa opioid receptors agonists.

Seizure threshold was determined by pentylenetetrazole (PTZ) (see FIG. 6) tail-vein infusion on freely moving pDyn deficient mice at a rate of 100 μl/min (100 μg/ml PTZ in saline, pH 7.4). Infusion was stopped when animals displayed generalized clonic seizures. The seizure threshold dose was calculated from the infused volume in relation to body weight. Dyn B or modified variants thereof were dissolved in DMSO and diluted to the final dosages in saline containing final concentrations of 10% DMSO, 3% Tween 80. 3 μl of peptide-solution were applied 30 min. before tail-vein infusion under mild sevoflurane anaesthesia into the cisterna magna. The following peptides were tested: Dyn B=SEQ ID No. 8, Dyn B G3A=SEQ ID 10 No. 11 wherein Z at position 3 is alanine, Dyn B G3A+Q8A=SEQ ID No. 11 wherein Z at position 3 is alanine and Z at position 8 of SEQ ID No. 11 is alanine, Dyn B Q8A=SEQ ID No. 11 wherein Z at position 8 is alanine.

1-20. (canceled)

21. A method for treating epilepsy comprising:
administering a delivery vector which provides activation of human Kappa Opioid receptors in the epileptogenic focus,
wherein the delivery vector comprises a DNA sequence comprising a sequence that encodes preprodynorphin or a preprodynorphin variant, wherein said sequence that encodes preprodynorphin or a preprodynorphin variant comprises a signal sequence that encodes a signal peptide,
wherein said pre-prodynorphin or pre-prodynorphin-variant comprises at least one of the following sequences:
a. Dyn A that is SEQ ID No. 7 (AA 207-223 of SEQ ID No. 1; ppDyn) or a variant thereof consisting of the first 13 AA (first from the N-terminal end) or a variant thereof consisting of the first 8 AA (first from the N-terminal end);
b. Dyn B that is SEQ ID No. 8 (AA 226-238 of SEQ ID No. 1; ppDyn);
c. leumorphin that is SEQ ID No. 9 (AA 226-254 of SEQ ID No. 1; ppDyn);
d. variants of Dyn A according to SEQ ID No. 7 having an amino acid sequence identity of at least 60% within the first 8 AA counted from the N-terminus of SEQ ID No. 7 (YGGFLRRI (SEQ ID NO: 18));
e. variants of Dyn B according to SEQ ID No. 8 having an amino acid sequence identity of at least 60% within the first 8 AA counted from the N-terminus of SEQ ID No. 8 (YGGFLRRQ (SEQ ID NO: 19));
f. variants of leumorphin according to SEQ ID No. 9 having an amino acid sequence identity of at least 60% within the first 8 AA counted from the N-terminus of SEQ ID No. 9 (YGGFLRRQ (SEQ ID NO: 19));
g. SEQ ID No. 10 (YGZFLRRZRPKLKWDNQ) wherein Z stands for any naturally occurring amino acid;
h. SEQ ID No. 11 (YGZFLRRZFKVVT) wherein Z stands for any naturally occurring amino acid; and
i. SEQ ID No. 12 (YGZFLRRZFKVVTRSQEDPNAYSGELFDA) wherein Z stands for any naturally occurring amino acid, and
releasing dynorphins or dynorphin-variants on demand from said delivery vector.

22. The method of claim 21 wherein said sequences are selected from variants having an amino acid sequence identity of at least 70% within the first 8 AA counted from the N-terminus of SEQ ID No. 7 (YGGFLRRI (SEQ ID NO: 18)), the N-terminus of SEQ ID No. 8 (YGGFLRRQ (SEQ ID NO: 19)), or the N-terminus of SEQ ID No. 9 (YGGFLRRQ (SEQ ID NO: 19)), respectively.

23. The method of claim 21 wherein said sequences are selected from variants having an amino acid sequence identity of at least 80% within the first 8 AA counted from the N-terminus of SEQ ID No. 7, the N-terminus of SEQ ID No. 8, or the N-terminus of SEQ ID No. 9, respectively.

24. The method of claim 21 wherein said delivery vector comprises a DNA sequence encoding a pre-prodynorphin-variant comprising at least one of the following sequences of variants selected from the group:

| SEQ ID No. 10 | |
|---|---|
| a) | (YGZFLRRZRPKLKWDNQ) |
| SEQ ID No. 11 | |
| b) | (YGZFLRRZFKVVT) |
| SEQ Id No. 12 | |
| c) | (YGZFLRRZFKVVTRSQEDPNAYSGELFDA), |

wherein Z stands for any naturally occurring amino acid.

25. The method of claim 21 wherein said delivery vector further comprises a recombinant adeno-associated virus (AAV) vector genome or further comprises a recombinant lentivirus genome.

26. The method of claim 21 wherein said delivery vector further comprises a recombinant adeno-associated virus (AAV) vector genome, wherein said recombinant adeno-associated virus (AAV) vector genome is a human serotype vector selected from serotypes 1, 2, 3, 4, 5, 6, 7, 8, 9, 10, rh10, 11, 12, 13, 14, serpentine AAV, ancestral AAV, and AAV capsid mutants derived thereof.

27. The method of claim 21 wherein the delivery vector is provided as a part of a recombinant virus particle or a liposome.

28. The method of claim 27 wherein said delivery vector further comprises a recombinant adeno-associated virus (AAV) vector genome and said recombinant adeno-associated virus vector genome is encapsidated in an adeno-associated virus capsid; or
said delivery vector further comprises a recombinant lentivirus vector genome and is packaged in a lentivirus particle.

29. A method of delivering a nucleic acid to a cell of the central nervous system, comprising:
contacting the cell with the delivery vector or recombinant virus particle or liposome of under conditions sufficient for the DNA sequence encoding pre-prodynorphin or pre-prodynorphin-variant to be introduced into the cell,
wherein the delivery vector drives expression of a pre-propeptide that is pre-prodynorphin or a pre-prodynorphin-variant wherein said pre-propeptide comprise the signal peptide and said signal peptide enables packing of a propeptide that is prodynorphin or a prodynorphin-variant, derived from said pre-propeptide, into vesicles,
wherein the delivery vector comprises a DNA sequence comprising a sequence that encodes preprodynorphin or a preprodynorphin variant, wherein said sequence that encodes preprodynorphin or a preprodynorphin variant comprises a signal sequence that encodes a signal peptide, and
wherein said pre-prodynorphin or pre-prodynorphin-variant comprises at least one of the following sequences:
j. Dyn A that is SEQ ID No. 7 (AA 207-223 of SEQ ID No. 1; ppDyn) or a variant thereof consisting of the first 13 AA (first from the N-terminal end) or a variant thereof consisting of the first 8 AA (first from the N-terminal end);
k. Dyn B that is SEQ ID No. 8 (AA 226-238 of SEQ ID No. 1; ppDyn);
l. leumorphin that is SEQ ID No. 9 (AA 226-254 of SEQ ID No. 1; ppDyn);
m. variants of Dyn A according to SEQ ID No. 7 having an amino acid sequence identity of at least 60% within the first 8 AA counted from the N-terminus of SEQ ID No. 7 (YGGFLRRI (SEQ ID NO: 18));
n. variants of Dyn B according to SEQ ID No. 8 having an amino acid sequence identity of at least 60% within the first 8 AA counted from the N-terminus of SEQ ID No. 8 (YGGFLRRQ (SEQ ID NO: 19));
o. variants of leumorphin according to SEQ ID No. 9 having an amino acid sequence identity of at least 60% within the first 8 AA counted from the N-terminus of SEQ ID No. 9 (YGGFLRRQ (SEQ ID NO: 19));
p. SEQ ID No. 10 (YGZFLRRZRPKLKWDNQ) wherein Z stands for any naturally occurring amino acid;
q. SEQ ID No. 11 (YGZFLRRZFKVVT) wherein Z stands for any naturally occurring amino acid; and
SEQ ID No. 12 (YGZFLRRZFKVVTRSQEDPNAYSGELFDA) wherein Z stands for any naturally occurring amino acid.

30. The method of claim 21 wherein the delivery vector is provided as a part of a pharmaceutical composition.

31. A method for treating focal epilepsy in a subject or for preventing epileptic seizures in a subject that suffers from focal epilepsy, comprising:
administering the delivery vector according to claim 1 to the subject, whereby said delivery vector provides activation of human Kappa Opioid Receptors in the epileptogenic focus, thereby inhibiting seizures.

32. The method according to claim 31, additionally comprising releasing by the delivery vector on-demand peptides with agonistic effects on human Kappa Opioid Receptors in the epileptogenic focus.

33. The method of claim 30, wherein said delivery vector is suitable for peripheral administration or for intracranial or for intracerebral or for intrathecal or for intraparenchymal administration.

34. The method according to claim 21, wherein said delivery vector is administered intracerebrally.

35. The method of claim 21, wherein the delivery vector additionally comprises a pharmaceutically acceptable carrier.

36. The method of claim 24, wherein at least one Z in a sequence according to a), b), or c) is a naturally occurring amino acid that differs from the corresponding amino acid of the wild-type sequence of said pre-prodynorphin-variant according to a sequence according to a), b), or c).

37. The method of claim 21, wherein said delivery vector comprises a DNA sequence encoding the signal peptide of SEQ ID No. 4.

38. The method of claim 21, wherein activation of human Kappa Opioid receptors in the epileptogenic focus is provided by driving expression of a pre-propeptide that is pre-prodynorphin or a pre-prodynorphin-variant wherein said pre-propeptide comprise the signal peptide and said signal peptide enables packing of a propeptide that is prodynorphin or a prodynorphin-variant, derived from said pre-propeptide, into vesicles.


SEQUENCE LISTING (24 sequences)

SEQ ID NO: 1 - protein, 254 aa, Homo sapiens [sequence omitted]

SEQ ID NO: 2 - protein, 274 aa, Homo sapiens
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQVKRYGGFLRRQFKVVTRSQEDPNAYSGELFDAKRSSEVAGEGDGDSMGHEDLYKRYGGFLRRIRPKLKWDNQKRYGGFLRRQFKVVTRSQEDPNAYSGELFDA

SEQ ID NO: 3 - protein, 286 aa, Homo sapiens
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQVKRYGGFLRRQFKVVTRSQEDPNAYSGELFDAKRSSEVAGEGDGDSMGHEDLYKRYGGFLRRQFKVVTRSQEDPNAYSGELFDAKRYGGFLRRQFKVVTRSQEDPNAYSGELFDA

SEQ ID NO: 4 - protein, 20 aa, Homo sapiens
MAWQGLVLAACLLMFPSTTA

SEQ ID NO: 5 - protein, 152 aa, Homo sapiens
DCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQV

SEQ ID NO: 6 - protein, 9 aa, Homo sapiens
YGGFLRKYP

SEQ ID NO: 7 - protein, 17 aa, Homo sapiens
YGGFLRRIRPKLKWDNQ

SEQ ID NO: 8 - protein, 13 aa, Homo sapiens
YGGFLRRQFKVVT

SEQ ID NO: 9 - protein, 29 aa, Homo sapiens
YGGFLRRQFKVVTRSQEDPNAYSGELFDA

SEQ ID NO: 10 - protein, 17 aa, Homo sapiens
  VARIANT 3: note = Any amino acid
  VARIANT 8: note = Any amino acid
YGXFLRRXRPKLKWDNQ

SEQ ID NO: 11 - protein, 13 aa, Homo sapiens
  VARIANT 3: note = Any amino acid
  VARIANT 8: note = Any amino acid
YGXFLRRXFKVVT

SEQ ID NO: 12 - protein, 29 aa, Homo sapiens
  VARIANT 3: note = Any amino acid
  VARIANT 8: note = Any amino acid
YGXFLRRXFKVVTRSQEDPNAYSGELFDA

SEQ ID NO: 13 - protein, 254 aa, Homo sapiens
  VARIANT 209: note = Any amino acid
  VARIANT 214: note = Any amino acid
  VARIANT 216: note = Any amino acid
  VARIANT 228: note = Any amino acid
  VARIANT 233: note = Any amino acid
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQVKRYGGFLRKYPKRSSEVAGEGDGDSMGHEDLYKRYGXFLRRXRXKLKWDNQKRYGXFLRRXFKVVTRSQEDPNAYSGELFDA

SEQ ID NO: 14 - protein, 274 aa, Homo sapiens
  VARIANT 177: note = Any amino acid
  VARIANT 182: note = Any amino acid
  VARIANT 229: note = Any amino acid
  VARIANT 234: note = Any amino acid
  VARIANT 236: note = Any amino acid
  VARIANT 248: note = Any amino acid
  VARIANT 253: note = Any amino acid
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQVKRYGXFLRRXFKVVTRSQEDPNAYSGELFDAKRSSEVAGEGDGDSMGHEDLYKRYGXFLRRXRXKLKWDNQKRYGXFLRRXFKVVTRSQEDPNAYSGELFDA

SEQ ID NO: 15 - protein, 286 aa, Homo sapiens
  VARIANT 177: note = Any amino acid
  VARIANT 182: note = Any amino acid
  VARIANT 229: note = Any amino acid
  VARIANT 234: note = Any amino acid
  VARIANT 260: note = Any amino acid
  VARIANT 265: note = Any amino acid
MAWQGLVLAACLLMFPSTTADCLSRCSLCAVKTQDGPKPINPLICSLQCQAALLPSEEWERCQSFLSFFTPSTLGLNDKEDLGSKSVGEGPYSELAKLSGSFLKELEKSKFLPSISTKENTLSKSLEEKLRGLSDGFREGAESELMRDAQLNDGAMETGTLYLAEEDPKEQVKRYGXFLRRXFKVVTRSQEDPNAYSGELFDAKRSSEVAGEGDGDSMGHEDLYKRYGXFLRRXFKVVTRSQEDPNAYSGELFDAKRYGXFLRRXFKVVTRSQEDPNAYSGELFDA

SEQ ID NO: 16 - DNA, 20 bp, synthetic construct
  misc_feature 1..20: note = Description of Artificial Sequence: Synthetic oligonucleotide
  source 1..20: mol_type = other DNA
ctagagctcgctgatcagcc

SEQ ID NO: 17 - DNA, 20 bp, synthetic construct
  misc_feature 1..20: note = Description of Artificial Sequence: Synthetic oligonucleotide
  source 1..20: mol_type = other DNA
tgtcttcccaatcctccccc

SEQ ID NO: 18 - protein, 8 aa, Homo sapiens
YGGFLRRI

SEQ ID NO: 19 - protein, 8 aa, Homo sapiens
YGGFLRRQ

SEQ ID NO: 20 - protein, 7 aa, synthetic construct
  REGION 1..7: note = Description of Artificial Sequence: Synthetic peptide
YGFLRRQ

SEQ ID NO: 21 - protein, 8 aa, synthetic construct
  REGION 1..8: note = Description of Artificial Sequence: Synthetic peptide
  VARIANT 3: note = Any amino acid
  VARIANT 8: note = Any amino acid
YGXFLRKX

SEQ ID NO: 22 - protein, 7 aa, synthetic construct
  REGION 1..7: note = Description of Artificial Sequence: Synthetic peptide
  VARIANT 7: note = Any amino acid
YGFLRKX

SEQ ID NO: 23 - protein, 8 aa, synthetic construct
  REGION 1..8: note = Description of Artificial Sequence: Synthetic peptide
  VARIANT 6..8: note = Any amino acid
YGGFLXXX

SEQ ID NO: 24 - protein, 8 aa, synthetic construct
  REGION 1..8: note = Description of Artificial Sequence: Synthetic peptide
  VARIANT 7: note = Any amino acid
YGAFLRXA